(19) United States
(12) Patent Application Publication
(10) Pub. No.: US 2024/0024661 A1
(43) Pub. Date: Jan. 25, 2024

(54) SYSTEMS, DEVICES, AND METHODS FOR TRANSCUTANEOUS ELECTRICAL STIMULATION

(71) Applicant: CEFALY Technology SPRL, Seraing (BE)

(72) Inventors: Jennifer TRAINOR-MCDERMOTT, Saint James, NY (US); Mickaël JEAN, Auboue (FR); Michael JOHNSON, Denver, CO (US); Jeroen WILMS, Seraing (BE); Michaël BURM, Seraing (BE); Robrecht Van VELTHOVEN, Seraing (BE); Laurent HOUTSAEGER, Seraing (BE)

(21) Appl. No.: 18/320,806

(22) Filed: May 19, 2023

Related U.S. Application Data
(60) Provisional application No. 63/343,904, filed on May 19, 2022.

(51) Int. Cl. A61N 1/04; A61N 1/36; G16H 20/40

(52) U.S. Cl. CPC A61N 1/0456; A61N 1/36171; A61N 1/36175; A61N 1/0472; A61N 1/36132; G16H 20/40

(57) ABSTRACT

Described here are systems, devices, and methods useful for treating a neurological disorder of a patient. A system for applying transcutaneous electrical stimulation to a patient may comprise an electrode configured to be coupled to a forehead of the patient. The electrode may comprise a substrate, a first conductor, a second conductor, and an insulator each disposed on the substrate. The insulator may be positioned laterally between the first and second conductor. The first and second conductors may be configured to stimulate a trigeminal nerve of the patient. An electrode identifier may be disposed on the substrate and across the first and second conductors. A housing may be configured to releasably couple to the electrode. The housing may comprise a signal generator configured to generate a set of pulses for the electrode.

### CROSS-REFERENCE TO RELATED APPLICATIONS

This application claims the benefit of U.S. Provisional Application No. 63/343,904, filed May 19, 2022, the content of which is hereby incorporated by reference in its entirety.

### TECHNICAL FIELD

Devices, systems, and methods herein relate to non-invasive transcutaneous electrical stimulation that may be used in therapeutic applications, including but not limited to treating a migraine.

### BACKGROUND

Migraine is a common neurobiological disorder characterized by recurrent episodes of headache accompanied by sensory hypersensitivity, which can significantly impair quality of life. Episodic migraine may be defined as a patient experiencing one to fourteen headache days per month with associated migraine symptoms (e.g., nausea, photophobia, phonophobia, etc.). Other disorders such as sleep disorders may also significantly reduce a patient's quality of life.

Conventional acute migraine treatments include pharmacological solutions such as analgesics, non-steroidal anti-inflammatory drugs (NSAIDs), and triptans. Similarly, pharmacological drugs are commonly used to treat disorders such as depression, anxiety, sleep, and the like. Conventional drugs are associated with several contraindications as well as moderate to severe side effects. For example, excessive consumption of acute migraine drugs may increase drug resistance and even increase migraine frequency. Accordingly, additional devices, systems, and methods of transcutaneous electrical stimulation may be desirable.

### SUMMARY

Described here are systems, devices, and methods useful for the acute non-invasive treatment of a disorder including but not limited to a migraine. Generally, a system for applying transcutaneous electrical stimulation to a patient may comprise an electrode configured to be coupled to a forehead of the patient. The electrode may comprise a substrate, a first conductor, a second conductor, and an insulator each disposed on the substrate. The insulator may be positioned laterally between the first and second conductors. The first and second conductors may be configured to stimulate a nerve of a patient, such as a trigeminal nerve. An electrode identifier may be disposed on the substrate and across the first and second conductors. A housing may be configured to releasably couple to the electrode. The housing may comprise a signal generator configured to generate a set of pulses for the electrode.

In some variations, the electrode identifier may comprise at least two apertures, a first magnet coupled to a first adhesive conductor, and a second magnet coupled to a second adhesive conductor. The first and second magnets may project through a respective aperture of the electrode identifier. In some of these variations, the housing may be configured to be coupled to the forehead of the patient using the electrode. In some of these variations, the housing may be configured to magnetically couple to the first and second magnets of the electrode. In some of these variations, the first and second magnets may be configured to receive the set of pulses generated by the signal generator. In some of these variations, the electrode identifier may define a third aperture and the insulator may define a fourth aperture corresponding to the third aperture of the insulator. In some variations, the electrode identifier may overlap the first and second conductors. In some variations, the electrode identifier may be disposed on the insulator. In some variations, the electrode identifier may overlap the insulator. In some variations, the electrode identifier may comprise a Radio Frequency Identification (RFID) tag. In some variations, the insulator may separate the first conductor from the second conductor. In some variations, the electrode may comprise an adhesive conductor area of the first and second adhesive conductor between about 50% and about 80% of a substrate area of the substrate.

In some variations, the first conductor may comprise a first lateral end opposite the insulator, and the second conductor may comprise a second lateral end opposite the insulator. In some of these variations, the first lateral end, the second lateral end, and the insulator may be non-overlapping with the first and second adhesive conductors. In some of these variations, the first lateral end and the second lateral end may comprise a lateral end area of up to about 20% of a substrate area of the substrate. In some of these variations, each of the first conductor and the second conductor may taper from the insulator to the respective lateral end.

In some variations, the signal generator may be configured to generate the set of pulses comprising a current of between about 1 mA and about 35 mA. In some variations, the signal generator may be configured to generate the set of pulses comprising a pulse width between about 240 μs and about 260 μs, a pulse amplitude of up to about 17 mA, and a dead time of between about 1 μs and about 10 μs. In some variations, the signal generator may be configured to generate the set of pulses comprising a duration of between about 150 microseconds and about 450 microseconds with a maximum increase in current of up to about 20 mA at a rate of less than or equal to about 40 microamperes per second and with a step up in current not exceeding about 50 microamperes.

In some variations, the electrode may be configured to stimulate an afferent path of a supratrochlear nerve and an afferent path of a supraorbital nerve of an ophthalmic branch of the trigeminal nerve. In some variations, the substrate may comprise a length of between about 70 mm and about 120 mm. In some variations, the insulator may comprise a length of between about 15 mm and about 50 mm, and a width of between about 5 mm and about 15 mm. In some variations, the first lateral end and the second lateral end may each comprise a height of between about 5 mm and about 20 mm, and a width of between about 5 mm and about 10 mm.

In some variations, the system may further comprise a power source. The housing may be configured to separate the power source from the signal generator. In some of these variations, the signal generator may be separated from the power source by a predetermined distance. In some of these variations, the housing may comprise a set of protrusions configured to separate the power source from the signal generator. In some of these variations, the power source may comprise a battery. In some variations, the housing may comprise a power source coupled to the signal generator. A charger may be configured to wirelessly charge the power source.

Also described here are variations of an electrode configured to be coupled to a forehead of the patient. The electrode may comprise a substrate, a first conductor, a second conductor, and an insulator each disposed on the substrate. The insulator may be positioned laterally between the first and second conductor. The first and second conductors may be configured to stimulate a trigeminal nerve of the patient. An electrode identifier may be disposed on the substrate and across the first and second conductors.

Also described here are variations of a device comprising a signal generator configured to generate a set of pulses for transcutaneous stimulation of a trigeminal nerve of a patient, an identifier reader configured to detect an electrode identifier of an electrode releasably coupled to the device, and a processor and a memory coupled to the identifier reader. The processor may be configured to detect the electrode identifier using the identifier reader, generate an authentication signal based on the detected identifier, and stimulate the trigeminal nerve of the patient using the set of pulses based on the authentication signal.

In some variations, the processor may be configured to inhibit generation of the set of pulses when the electrode identifier is not detected. In some variations, the processor may be configured to inhibit generation of the set of pulses when the authentication signal is one or more of unauthorized, expired, and used.

Also described here are methods of treating a patient comprising coupling an electrode to a forehead of the patient, the electrode comprising an electrode identifier for the electrode, coupling a housing of an electrical stimulation device to the electrode, the electrical stimulation device comprising a signal generator and an identifier reader, detecting the electrode identifier using the identifier reader of the electrical stimulation device, generating an authentication signal based on the detected identifier, and stimulating a trigeminal nerve of the patient using a set of pulses generated by the signal generator based on the authentication signal.

In some variations, the stimulating may be configured to treat one or more of migraine, tension, headaches, cluster headaches, hemicrania continua, Semi Unilateral Neuralgaform Non Conjunctival Tearing (SUCNT), chronic paroxystic hemicranias, trigeminal neuralgia, facial nerve disturbances, autism, depression, cyclothymia, coma, anxiety, tremor, aphasia, insomnia, sleep disorders, hypersomnia, epilepsy, attention deficit hyperactivity disorder, Parkinson's disease, Alzheimer's disease, multiple sclerosis, stroke, and Cerebellar syndrome. In some variations, the method may further comprise releasing the device from the electrode. In some variations, the method may further comprise storing one or more of a session time, a treatment stimulation program selected, a session duration, a maximum current amplitude in a session, a session error, a number of repetitions, a sum of current delivered, a sum of current delivered if maximum current amplitude was reached, a battery charge time, a battery charge duration, a duration to reach full charge, and a battery charge error.

Also described here are methods for applying transcutaneous electrical stimulation to a patient comprising selecting one or more stimulation parameters for the electrical stimulation, applying the electrical stimulation having the selected one or more stimulation parameters using an electrical stimulation system coupled to the patient, determining a dosage of the electrical stimulation applied to patient, and modifying at least one stimulation parameter based on the determined dosage.

In some variations, determining the dosage may comprise calculating an electric charge delivered to the patient by the electrical stimulation system. In some variations, selecting the one or more stimulation parameters may comprise selecting one of a first treatment program having a first set of stimulation parameters and configured to preemptively treat a disorder and a second treatment program having a second set of stimulation parameters and configured to acutely treat the disorder.

In some variations, modifying the at least one stimulation parameter may be based on the determined dosage comprising increasing a first treatment program session frequency and reducing a second treatment program session frequency. In some variations, modifying the at least one stimulation parameter may be based on the determined dosage results in increasing the dosage of the first treatment program. In some variations, the dosage may be reduced over a predetermined time period after modifying the at least one stimulation parameter.

In some variations, a stimulation parameter of the one or more stimulation parameters may be adjusted while applying the electrical stimulation. In some variations, a third treatment program may be generated having a third set of stimulation parameters based on the adjusted stimulation parameter. In some variations, selecting the one or more stimulation parameters may comprise selecting the third treatment program. In some variations, a graphical user interface may be generated comprising the determined dosage.

In some variations, the one or more stimulation parameters may comprise one or more of a frequency, a current, a pulse width, a pulse amplitude, a dead time, a pulse duration, a session time, a session duration, a maximum current amplitude in a session, and a session frequency. In some variations, the electrical stimulation may comprise a frequency of the electrical stimulation, wherein the frequency is between about 10 Hz and about 300 Hz. In some variations, the electrical stimulation may comprise a current of between about 1 mA and about 35 mA. In some variations, the electrical stimulation may comprise a pulse width between about 240 μs and about 260 μs. In some variations, the electrical stimulation may comprise a pulse amplitude of up to about 17 mA. In some variations, the electrical stimulation may comprise a dead time of between about 1 μs and about 10 μs. In some variations, the electrical stimulation may comprise a duration of between about 150 microseconds and about 450 microseconds with a maximum increase in current of up to about mA at a rate of less than or equal to about 40 microamperes per second and with a step up in current not exceeding about 50 microamperes.

In some variations, applying the electrical stimulation may comprise stimulating an afferent path of a supratrochlear nerve and an afferent path of a supraorbital nerve of an ophthalmic branch of a trigeminal nerve. In some variations, the electrical stimulation system may comprise a signal generator releasably coupled to an electrode. Applying the electrical stimulation may comprise generating a set of pulses for the electrode using the signal generator. In some variations, applying the electrical stimulation may treat one or more of: a migraine, tension, headaches, cluster headaches, hemicrania continua, Semi unilateral neuralgaform non conjunctival tearing (SUCNT), chronic paroxystic hemicranias, trigeminal neuralgia, facial nerve disturbances, autism, depression, cyclothymia, coma, anxiety, tremor, aphasia, insomnia, sleep disorders, hypersomnia, epilepsy, attention deficit hyperactivity disorder, Parkinson's disease, Alzheimer's disease, multiple sclerosis, stroke, and Cerebellar syndrome.

Also described here are electrical stimulation systems comprising an electrode configured to be coupled to a patient, a signal generator operably coupled to the electrode and configured to generate a set of pulses for transcutaneous electrical stimulation of the patient, and a processor and a memory coupled to the signal generator. The processor may be configured to receive one or more stimulation parameters, apply the electrical stimulation having the received one or more stimulation parameters to a nerve of a patient using the electrode, determine a dosage of the electrical stimulation applied to the nerve, and receive at least one modified stimulation parameter based on the determined dosage.

In some variations, determining the dosage may comprise calculating an electric charge delivered to the nerve of the patient. In some variations, receiving the one or more stimulation parameters may comprise selecting one of a first treatment program having a first set of stimulation parameters and configured to preemptively treat a disorder and a second treatment program having a second set of stimulation parameters and configured to acutely treat the disorder.

In some variations, the processor may be configured to receive an increase in a first treatment program session frequency based on the determined dosage and a reduction in a second treatment program session frequency. In some variations, the processor may be configured to receive an increase in the dosage of the first treatment program. In some variations, the processor may be configured to receive a reduction in the dosage of the first treatment program over a predetermined time period after modifying the at least one stimulation parameter.

In some variations, the processor may be configured to receive at least one modified stimulation parameter during one of the first treatment session and the second treatment session. In some variations, the processor may be configured to generate a third treatment program having a third set of stimulation parameters based on the received at least one modified stimulation parameters during one of the first treatment session and the second treatment session.

In some variations, the processor may be configured to receive a selection of the third treatment program. In some variations, the processor may be configured to generate a graphical user interface comprising the determined dosage.

In some variations, the electrical stimulation may comprise one or more of a frequency, a current, a pulse width, a pulse amplitude, a dead time, a pulse duration, a session time, a session duration, a maximum current amplitude in a session, and a session frequency. In some variations, the electrical stimulation may comprise a frequency of between about 10 Hz and about 300 Hz. In some variations, the electrical stimulation may comprise a current of between about 1 mA and about 35 mA. In some variations, the electrical stimulation may comprise a pulse width between about 240 μs and about 260 μs. In some variations, the electrical stimulation may comprise a pulse amplitude of up to about 17 mA. In some variations, the electrical stimulation may comprise a dead time of between about 1 μs and about 10 μs. In some variations, the electrical stimulation may comprise a duration of between about 150 microseconds and about 450 microseconds with a maximum increase in current of up to about 20 mA at a rate of less than or equal to about 40 microamperes per second and with a step up in current not exceeding about 50 microamperes.

### BRIEF DESCRIPTION OF THE DRAWINGS

FIG. 1 is a schematic block diagram of an illustrative variation of a transcutaneous electrical stimulation system.

FIGS. 2A-2C are front views of illustrative variations of stimulation systems coupled to the forehead of a patient.

FIG. 3A is a front perspective view of an illustrative variation of a transcutaneous electrical stimulation system. FIG. 3B is a rear perspective view of an illustrative variation of a transcutaneous electrical stimulation system. FIG. 3C is a front view of an illustrative variation of a transcutaneous electrical stimulation system. FIG. 3D is a rear view of an illustrative variation of a transcutaneous electrical stimulation system. FIG. 3E is a side view of an illustrative variation of a transcutaneous electrical stimulation system. FIG. 3F is a top view of an illustrative variation of a transcutaneous electrical stimulation system.

FIG. 4 is an exploded view of an illustrative variation of a transcutaneous electrical stimulation system.

FIGS. 5A-5C are schematic plan views of illustrative variations of an electrode.

FIG. 6A is a schematic plan view of an illustrative variation of an electrode. FIG. 6B is a schematic cross-sectional plan view of an illustrative variation of the electrode depicted in FIG. 6A.

FIG. 6C is a schematic cross-sectional side view of an illustrative variation of the electrode depicted in FIG. 6B.

FIG. 7 is a schematic plan view of an illustrative variation of an electrode.

FIGS. 8A and 8B are schematic exploded perspective views of illustrative variations of an electrode.

FIG. 9 is a rear exploded perspective view of an illustrative variation of a connector of a transcutaneous electrical stimulation system.

FIGS. 10A-10C are schematic side views of illustrative variations of a magnet of a transcutaneous electrical stimulation system.

FIG. 11 is a schematic plan view of an illustrative variation of an RFID circuit of a transcutaneous electrical stimulation system.

FIG. 12A is a front perspective view of an illustrative variation of a switch of a transcutaneous electrical stimulation system. FIG. 12B is a side view of an illustrative variation of a switch of a transcutaneous electrical stimulation system. FIG. 12C is a rear view of an illustrative variation of a switch of a transcutaneous electrical stimulation system.

FIG. 13A is a front perspective view of an illustrative variation of a rear housing of a transcutaneous electrical stimulation system. FIG. 13B is a front view of an illustrative variation of a rear housing of a transcutaneous electrical stimulation system.

FIG. 14A is a front perspective view of an illustrative variation of a wireless charger. FIG. 14B is a front view of an illustrative variation of a wireless charger. FIG. 14C is a rear view of an illustrative variation of a wireless charger. FIG. 14D is a side view of an illustrative variation of a wireless charger.

FIGS. 15A and 15B are plots of illustrative variations of pulse waveforms for transcutaneous electrical stimulation. FIG. 15C is a plot of stimulation intensity over time.

FIG. 16 depicts a flowchart representation of an illustrative variation of electrode authentication.

FIGS. 17A-17G depict illustrative variations of a graphical user interface relating to a transcutaneous electrical stimulation interface.

FIGS. 18A-18L depict illustrative variations of a graphical user interface relating to a transcutaneous electrical stimulation.

FIGS. 19A-19G depict illustrative variations of a graphical user interface relating to patient data.

FIG. 20 depicts a flowchart representation of an illustrative variation of applying neurostimulation.

### DETAILED DESCRIPTION

Described here are systems, devices, and methods for non-invasive transcutaneous electrical stimulation such as for the electrotherapeutic treatment of a disorder. For example, the systems, devices, and methods described herein may improve treatment efficacy, patient compliance, ease of use, and/or optimize dosage by, for example: providing an ergonomic electrode configuration; providing electrode authentication to prevent patient error and/or unauthorized use; magnetically coupling a durable housing component to a disposable electrode component to facilitate electrode positioning and system setup; improving system performance based on component layout; determining neurostimulation dosage; modifying stimulation parameters for different treatment goals; and/or achieving an optimal dose (e.g., lowest effective dose). For example, an optimal dose for some patients may minimize or achieve a lowest effective dose.

The systems, devices, and methods described herein may transcutaneously stimulate one or more nerves of a patient. In some variations, the systems, devices, and methods described herein may transcutaneously stimulate an ophthalmic branch of the trigeminal nerve of a patient, thereby generating an analgesic effect to treat the patient. For example, a set of electrodes may be configured to deliver a set of pulses to electrically stimulate the nerve endings of the trigeminal nerve. The trigeminal nerve includes an ophthalmic branch, a maxillary branch, and a mandibular branch. The trigeminal nerve on the forehead divides into the internal frontal (or supratrochlear) nerve and the external frontal (or supraorbital) nerve. The set of pulses may be delivered to the patient transcutaneously via the supraorbital electrode to excite (e.g., trigger action potentials) the afferent paths of the supratrochlearis and supraorbitalis (or supratrochlear and supraorbital) nerves belonging to an upper branch of the trigeminal nerve (V1). The therapeutic effect of the treatment may be sustained for at least 24 hours after treatment.

The systems, device, and methods may thus stimulate the ophthalmic branch of the nerve of a patient, thereby generating an analgesic effect to treat the patient. For example, stimulation may treat one or more of migraine, tension, headaches, cluster headaches, hemicrania continua, Semi Unilateral Neuralgaform Non Conjunctival Tearing (SUCNT), chronic paroxystic hemicranias, trigeminal neuralgia, facial nerve disturbances, autism, depression, cyclothymia, coma, anxiety, tremor, aphasia, insomnia, sleep disorders, hypersomnia, epilepsy, attention deficit hyperactivity disorder, Parkinson's disease, Alzheimer's disease, multiple sclerosis, stroke, and Cerebellar syndrome.

In some variations, with respect to treating a migraine, the set of pulses having a relatively higher frequency may be configured to induce a sedative effect on the nervous system to provide pain relief to a patient during a migraine. A set of pulses applied at predetermined intervals (e.g., over a number of sessions across different days) may be configured to stimulate the central nervous system to treat the fronto-temporal cortex of a patient and reduce the frequency of a migraine.

In some variations, the systems, device, and methods for the electrotherapeutic treatment of a disorder may provide a first treatment configured to preemptively treat a disorder (e.g., preventative treatment) and a second treatment configured to acutely treat a disorder (e.g., acute treatment). For example, an objective of a first migraine treatment may be to reduce the frequency of and/or shorten the duration of future migraines while an objective of a second migraine treatment may be to reduce the intensity of and/or shorten the duration of a presently occurring migraine. The first migraine treatment may be provided while a patient is migraine-free (is not currently experiencing a migraine), while the second migraine treatment may be provided while the patient is experiencing a migraine (e.g., upon first symptoms of a migraine).

Generally, prioritization and excessive use of acute neurostimulation (e.g., second migraine treatment) over preventative neurostimulation (e.g., first migraine treatment) may increase a risk of adverse effects compared to elective preventative treatments that are generally better tolerated than acute neurostimulation. For example, the total energy delivered during the second migraine treatment may be higher than the total energy delivered during the first migraine treatment. Accordingly, it may be beneficial to track stimulation parameters over time, determine applied doses of electrical stimulation, and modify the stimulation parameters of a neurostimulation treatment program to optimize the therapy provided. For example, in some variations, optimization of the therapy may include achieving an optimal dose (e.g., lowest effective dose) (the lowest concentration of the therapy) needed in order to achieve a desired effect and minimize risk of adverse effects. Furthermore, pain tolerance for neurostimulation may vary from patient to patient (especially when starting a neurostimulation treatment protocol) due to individual anatomical differences and central pain mechanism variability. Perception of neurostimulation intensity may change over time due to the complex processes of acclimation, allodynia, and habituation. However, a standard dosing metric for neurostimulation is not conventionally known such that neurostimulation dosage determination has presently been limited to oversimplified (e.g., incomplete) proxies (e.g., duration of treatment, number of treatments), thereby limiting optimization of neurostimulation treatments. Analyzing and modifying neurostimulation treatments based on a dosage metric may facilitate an optimized dose (e.g., lowest effective dose) and an improvement in patient outcomes.

In some variations, a patient may increase the frequency (e.g., number of treatment sessions per time period) and intensity (e.g., maximum pulse amplitude) of a first treatment over time to achieve greater preemptive treatment benefits. For example, as the number, duration, and/or intensity of first treatments increases, the frequency and/or intensity of migraines may decrease, thereby reducing the number of second treatments performed, and thereby optimizing the therapy by achieving an optimal dose (e.g., lowest effective dose).

I. Systems and Devices

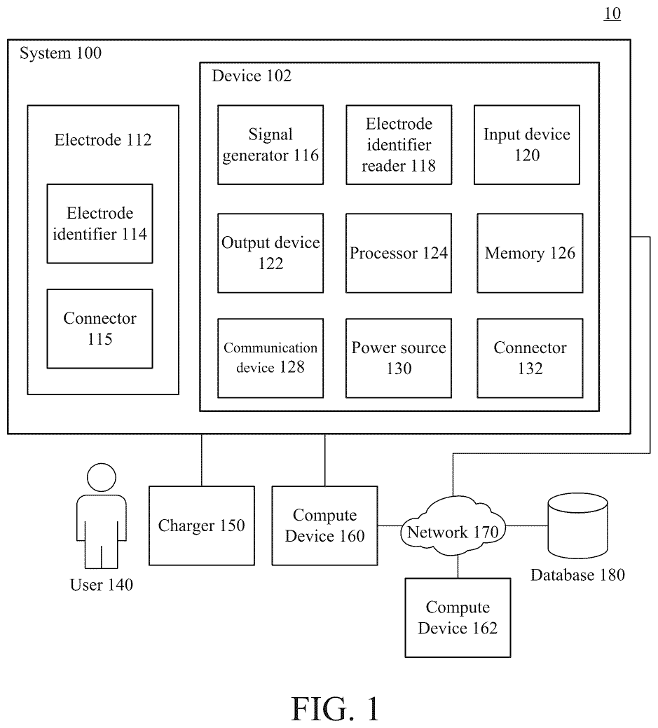

Generally, a transcutaneous electrical stimulation system may include one or more of the components necessary to treat and optionally monitor a patient using the systems as described herein. A block diagram of an exemplary transcutaneous electrical stimulation system 10 is depicted in FIG. 1. The system 10 may comprise one or more of a wearable system 100 (e.g., stimulator, external nerve stimulator, electrotherapy device), a charger 150, a compute device(s) 160, 162, and a database 180. In some variations, the wearable system 100 may be coupled to one or more of the compute device(s) 160, 162, and the database 180 via a network 170 (e.g., via a wired or wireless connection). The wearable system 100 may be configured to apply external nerve stimulation, such as external trigeminal nerve stimulation (e-TNS) to treat a patient. For example, the wearable system 100 may be configured to treat a disorder in a non-invasive manner. For example, the wearable system 100 may be configured to deliver a set of pulses to one or more of the supraorbital and supratrochlear nerves of the ophthalmic branch of the trigeminal nerve to generate an analgesic effect. In some variations, the wearable system 100 may be releasably coupled to a forehead of the patient to apply external neurostimulation to treat a disorder.

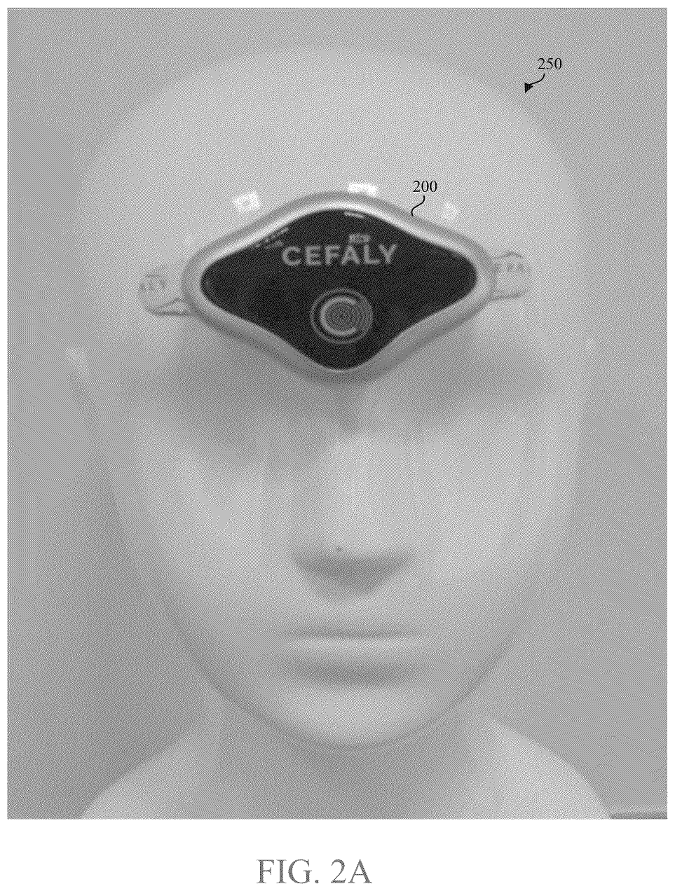

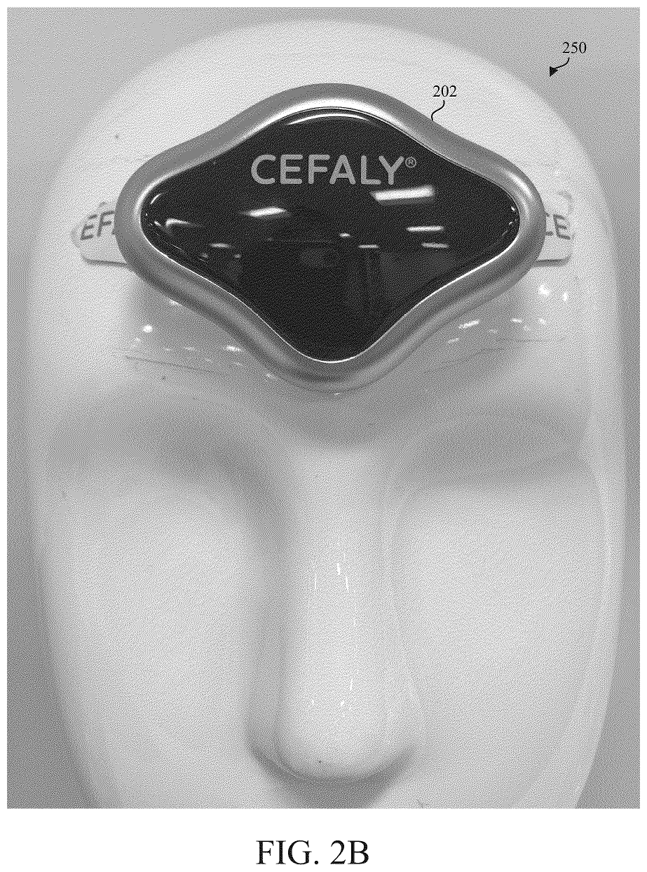

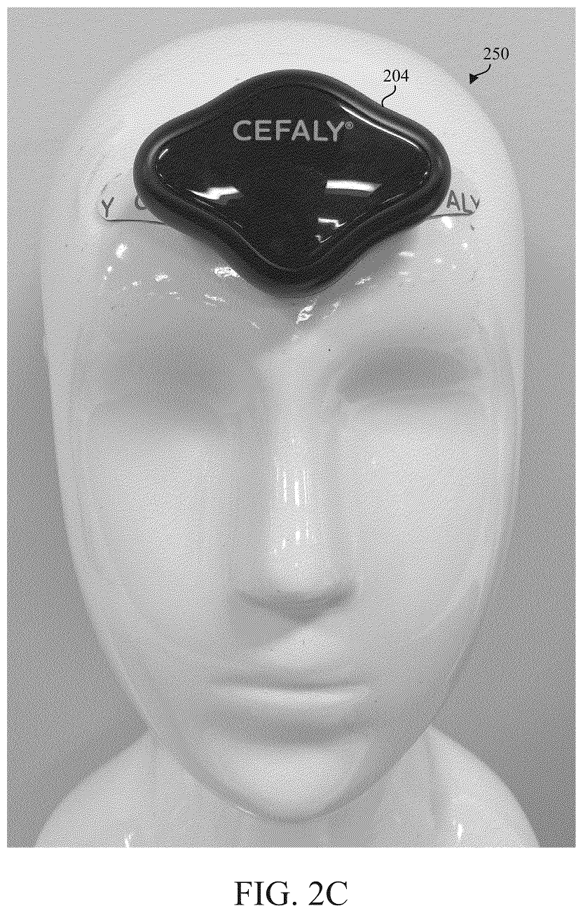

FIGS. 2A-2C depict respective wearable systems 200, 202, 204 having similar features and/or components as wearable system 100, releasably coupled to a forehead of a patient 250. In some variations, an electrode of the system may be configured to releasably couple (e.g., adhere using, e.g., an adhesive) to a forehead of a patient such as in a supraorbital region, which typically has a low amount of hair, to facilitate self-supported coupling and release of the electrode to the forehead. For example, the electrode may be coupled to a strap or band (e.g., elastic strap or loop, headband) configured to be held in place on and/or around a patient's head. Each of the systems 200, 202, 204 may be self-supported on the forehead for at least as long as a treatment session (e.g., up to about 20 minutes, up to about 60 minutes, up to about 120 minutes). As described in more detail herein, the system may comprise a durable component (e.g., a housing) including a signal generator and a processor, and a disposable component (e.g., an electrode). The disposable component may be manually coupled and decoupled to the patient (e.g., supraorbital region of the forehead), and the durable component may be manually coupled and decoupled from the disposable component (e.g., electrode) thereby releasably coupling the system to the patient. In some variations, wearable systems 200 corresponds to a variation without an electrode identifier, wearable system 202 corresponds to a variation including an electrode identifier, and wearable system 204 corresponds to a variation including an electrode identifier and configured to be controlled using an external device (e.g., compute device, smartphone) using the GUIs described herein.

Referring back to FIG. 1, in some variations, the wearable system 100 may comprise one or more of an electrode 112, which may comprise an electrode identifier 114 and a connector 115, and a stimulation device 102, which may comprise a signal generator 116, an electrode identifier reader 118, an input device 120, an output device 122, a processor 124, a memory 126, a communication device 128, a power source 130, and a connector 132, each of which are described in more detail herein.

In some variations, the wearable system 100 may comprise a durable component and a disposable component. For example, a disposable component of the wearable system 100 may include the electrode 112. In some variations, the durable component of the wearable system 100 may comprise a stimulation device 102 configured to carry, enclose, or otherwise contain one or more of the signal generator 116, the electrode identifier reader 118, the input device 120, the output device 122, the processor 124, the memory 126, the communication device 128, the power source 130, and the connector 132. The stimulation device 102 may be a reusable portion of the wearable system 100 while the electrode 112 may be disposable and replaced after a predetermined number of uses and/or time intervals (e.g., single use, limited use). Accordingly, the electrode 112 may be releasably coupled to the stimulation device 102. In some variations, the stimulation device 102 may be provided separately from the electrode 112. The durable component may provide long-term functionality given proper maintenance (e.g., cleaning, charging). In some variations, the housing of the stimulation device may be formed by, for example, one or more of injection molding, machining, solvent bonding, interference/press fit assembly, ultrasonic welding, and additive manufacturing (e.g., 3D printing) techniques. One or more of the components of the wearable system 100 may be disposed on a printed circuit board (PCB).

In some variations, the electrode 112 may be configured to releasably (e.g., reversibly) couple, adhere, and/or attach to a forehead of a patient for delivery of transcutaneous electrical stimulation. For example, the electrode 112 may be releasably coupled to the stimulation device 102 via the connector 132 (e.g., magnet), as described in more detail herein. In this manner, the electrode 112 may be adhered (e.g., via adhesive) to the forehead of the patient and the stimulation device 102 may be separately coupled to the electrode 112. In some variations the electrode 112 may comprise the electrode identifier 114. The electrode identifier 114 may be configured to uniquely identify and/or authenticate the electrode 112 as compatible and/or authorized for use as a component of the wearable system 100. This may increase patient safety and treatment efficacy by preventing usage of the wearable system 100 with unauthorized and/or expired electrodes. In some variations, the electrode identifier 114 may comprise a Radio Frequency Identification (RFID) tag. The electrode identifier 114 may be detected by a corresponding electrode identifier reader 118 disposed in the stimulation device 102.

In some variations, the signal generator 116 may be configured to generate a set of electrical pulses based on a set of stimulation parameters as described in more detail herein. In some variations, the input device 120 may be configured to generate an input signal (e.g., start/stop treatment) based on patient input (e.g., button press). In some variations, the output device 122 may be configured to output data (e.g., visual and/or auditory notifications) associated with the usage of the wearable system 100. In some variations, the processor 124 and memory 126 may be configured to control the wearable system 100. In some variations, the communication device 128 may be configured to communicate with one or more components of the system 10 such as the network 170, the compute device(s) 160, 162 (e.g., mobile phone, tablet, laptop, desktop PC), and database 180. The power source 130 may be configured to provide energy to one or more components of the wearable system 100. For example, the power source 130 may be a battery (e.g., rechargeable or non-rechargeable) or other source of energy. In some variations, the power source 130 may be coupled to and recharged by a charger 150 via a wired and/or wireless connection.

In some variations, the wearable system 100 may comprise a signal generator 116 configured to generate a set of pulses for transcutaneous stimulation of a trigeminal nerve of a patient. As mentioned above, an identifier reader 118 may be configured to detect an electrode identifier 114 of an electrode 112 releasably coupled to the wearable system 100. A processor 124 and a memory 126 may be coupled to the identifier reader 118. The processor 124 may be configured to detect the electrode identifier 114 using the identifier reader 118, generate an authentication signal based on the detected identifier 114, and stimulate the patient 140 (e.g., trigeminal nerve) using the set of pulses based on the authentication signal. In some variations, the processor may be configured to inhibit generation of the set of pulses when the electrode identifier 114 is not detected. In some variations, the processor may be configured to inhibit generation of the set of pulses when the authentication signal is one or more of unauthorized, expired, and used.

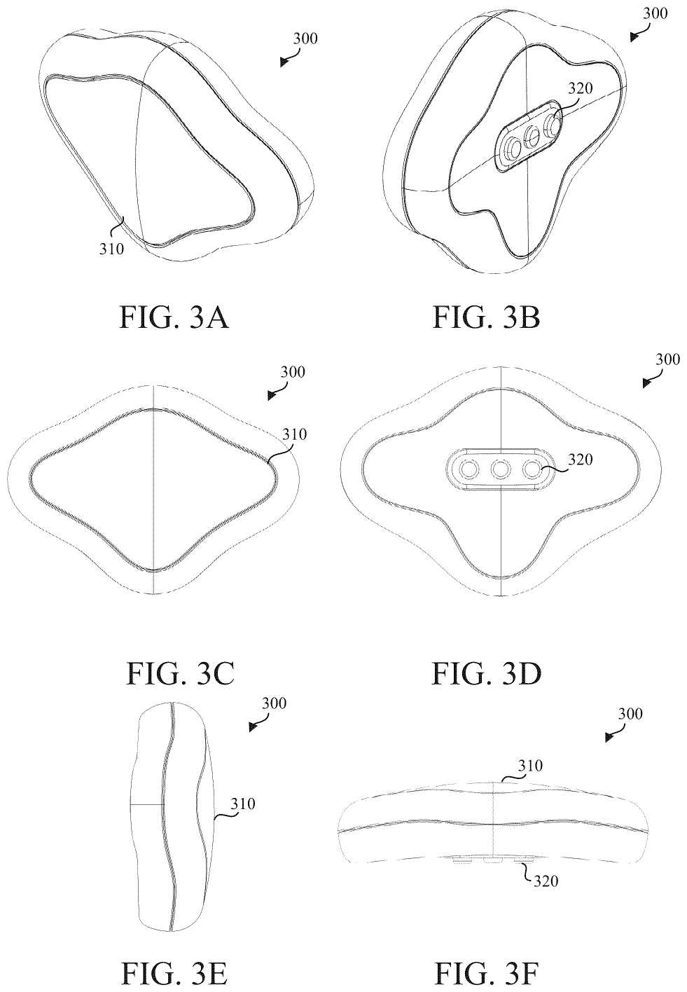

FIGS. 3A-3F are various exterior views (e.g., perspective, front, rear, side, top) of a durable component (e.g., housing) of a transcutaneous electrical stimulation system 300. A first side (e.g., front) of the system 300 may comprise an input device 310 (e.g., button, switch) configured to receive input (e.g., instructions) from a patient to facilitate operating the system 300. A second side (e.g., rear) of the system 300 opposite the first side may comprise a connector 320 configured to couple to a corresponding connector of a disposable electrode (not shown for the sake of clarity). When coupled to the forehead of the patient, the first side may face away from the patient and the second side may face toward the patient. In some variations, the connector 320 may be configured to electrically connect an electrode (not shown in FIGS. 3A-3F) to an internal signal generator of the system 300. Additionally or alternatively, the connector 320 may be configured to mechanically and/or magnetically couple the electrode to the system 300. For example, the connector 320 may comprise a snap button and/or a set of magnets.

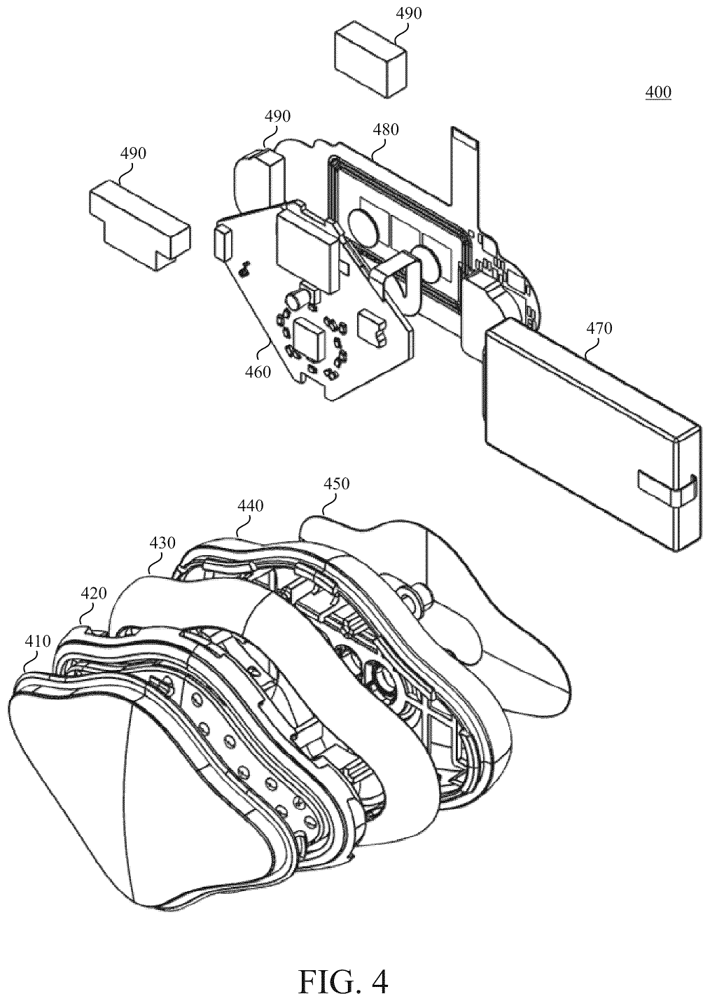

FIG. 4 is an exploded view of an illustrative variation of a transcutaneous electrical stimulation system 400 comprising a switch cover 410, a switch 420, a front housing 430, a rear housing 440, a label 450, an electronic circuit 460 (e.g., signal generator, processor, memory), a power source 470, an electrode identifier reader 480, and one or more spacers 490. Each of these components are described in more detail herein. From a first side (e.g., switch cover 410, front of system 400) to a second side (e.g., rear housing 440, rear of system 400) of the system 400, the switch 420, the electronic circuit 460, the power source 470 and the spacers 490, and the electrode identifier reader 480 may be sequentially disposed within the front housing 430 and rear housing 440. For the sake of clarity, the electronic circuit 460, power source 470, spacers 490, and electrode identifier reader 480 are shown in FIG. 4 as removed from the front housing 430 and rear housing 440.

In some variations, the housing of the transcutaneous electrical stimulation system may have a length of between about 50 mm and about 75 mm, a width of between about 30 mm and about 60 mm, and a depth of between about 10 mm and about 20 mm, including all ranges and sub-values in-between. In some variations, a housing of the system may be composed of one or more of thermoplastic polymers, such as, for example, polycarbonate/acrylonitrile butadiene styrene (PC/ABS) and polycarbonate (PC).

A. Electrode

Generally, the electrodes described herein may be configured to releasably couple to a forehead of a patient for the delivery of a set of pulses such as biphasic electrical pulses. For example, the electrode may include an adhesive having two patient contact portions configured to adhere to the skin and two system contact portions configured to receive the set of pulses from a stimulation system (e.g., signal generator).

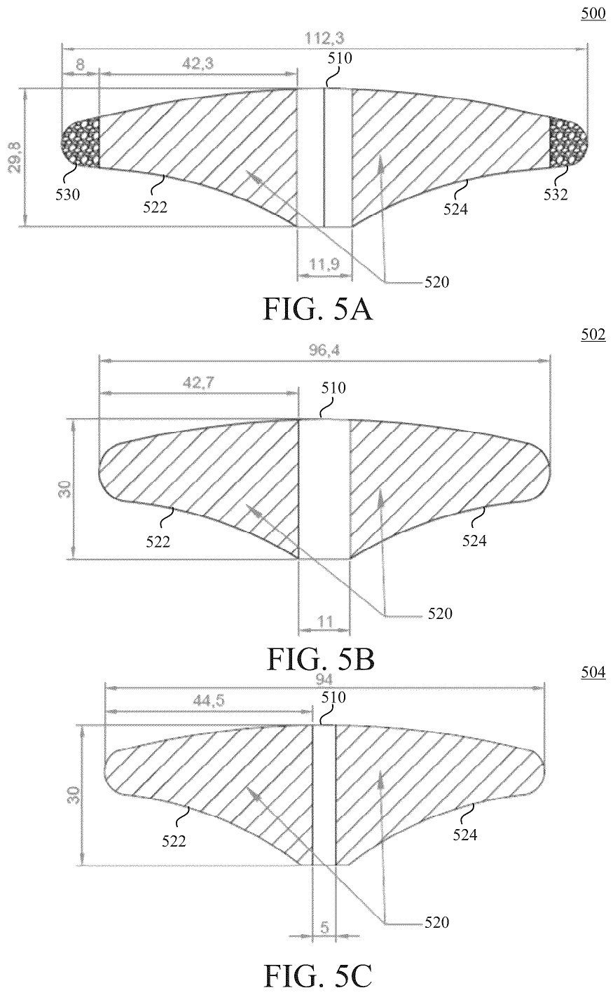

FIGS. 5A-5C are schematic views of illustrative variations of respective electrodes 500, 502, 504. Generally, electrode 500 is narrower and longer than electrodes 502, 504 to accommodate lateral ends 530, 532 while maintaining a similar total area. Electrode 502 has a wider insulator 510 and shallower taper than electrode 504. An electrode 500 may comprise an insulator 510 and a conductor 520 including a first conductor 522 and a second conductor 524. The insulator 510 separates the first conductor 522 from the second conductor 524. Each of the first conductor 522 and the second conductor 524 may taper from the insulator 510 to the respective lateral end 530, 532 of the electrode 500. The tapered configuration of the electrode 500, 502, 504 may ensure only the desired nerves are stimulated, as well as facilitate patient handling of the electrode. Electrodes 500, 502, 504 are depicted with conductors having the same area (e.g., about 1600 mm²). In some variations, the first conductor 522 may comprise a first lateral end 530 opposite the insulator 510, and the second conductor 524 may comprise a second lateral end 532 opposite the insulator 510. In some variations, the first lateral end 530, the second lateral end 532, and the insulator 510 are non-overlapping with the first and second adhesive conductors 522, 524. In some variations, the first lateral end 530 and the second lateral end 532 may each comprise a lateral end area of up to about 20% of an area of the electrode 500. In some variations, the lateral ends 530, 532 may be absent adhesive and facilitate handling by a user (e.g., patient). While only electrode 500 explicitly depicts conductors 522, 524 having lateral ends 530, 532, any of electrodes 502, 504 and those described herein may comprise the lateral ends described herein.

In some variations, the electrode may comprise a length of between about 90 mm and about 120 mm, and a height of between about 25 mm and about 35. In some variations, a conductor of the electrode comprise a length of between about 35 mm and about 60 mm, and a height of between about 25 mm and about 35 mm. In some variations, an insulator of the electrode may comprise a length of between about 2 mm and about 15 mm.

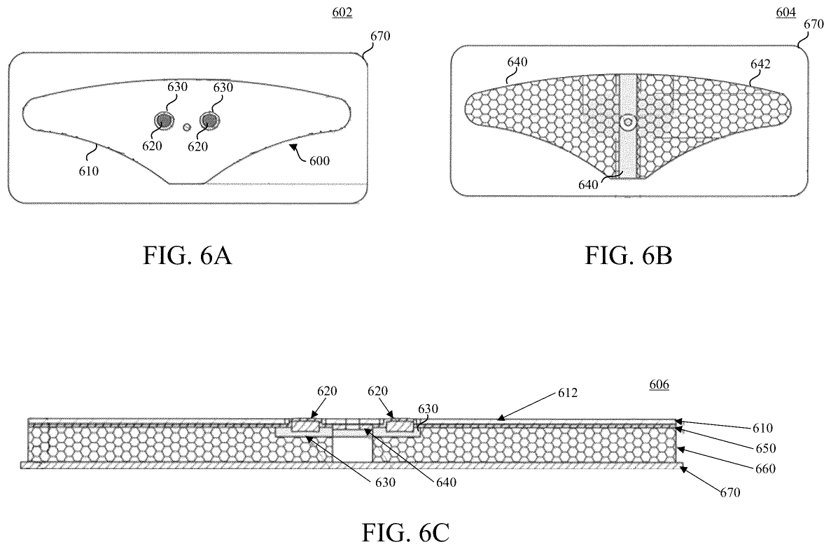

FIGS. 6A-6C illustrate a respective schematic plan view 602, cross-sectional plan view 604, and cross-sectional side view 606 of a variation of an electrode 600. The electrode 600 may comprise a plurality of layers including a substrate 610, a set of connectors 620 (e.g., two, three, four or more), one or more connector substrates 630, and an insulator 640, a conductor 650, an adhesive conductor 660, and a release liner 670. In some variations, the substrate 610 may comprise a metal surface 612 and/or a backing material. In some variations, the set of connectors 620 may comprise a set of plates, magnets, and/or fasteners. In some variations, the connector substrate 630 may be composed of a thermoplastic polymer including, for example, polyvinyl chloride, (PVC), polyethylene terepthalate (PET), nylon, urethane, polyethylene (PE), combinations thereof, and the like. In some variations, the connector substrate 630 may comprise an electrode identifier as described in more detail herein. In some variations, the insulator 640 may comprise a non-woven fabric. In some variations, the adhesive conductor 660 may comprise a gel.

In some variations, the set of connectors 620 (e.g., metal plates) may be circular and may have a radius of about 4 mm and a thickness of between about 0.4 mm and about 1 mm, including all ranges and sub-values in-between. In some variations, the set of connectors 620 may have a diameter (e.g., length, width) of about 8 mm. In some variations, a center-to-center distance between adjacent connectors of the set of connectors 620 may be about 13 mm, a width of an insulator 640 may be about 7 mm and a length of the insulator 640 may be about 43 mm, a width of the electrode 600 may be about 43 mm and a length of the electrode 600 may be about 94 mm. In some variations, a conductor 650 may comprise an area of between about 1500 mm² and about 1700 mm².

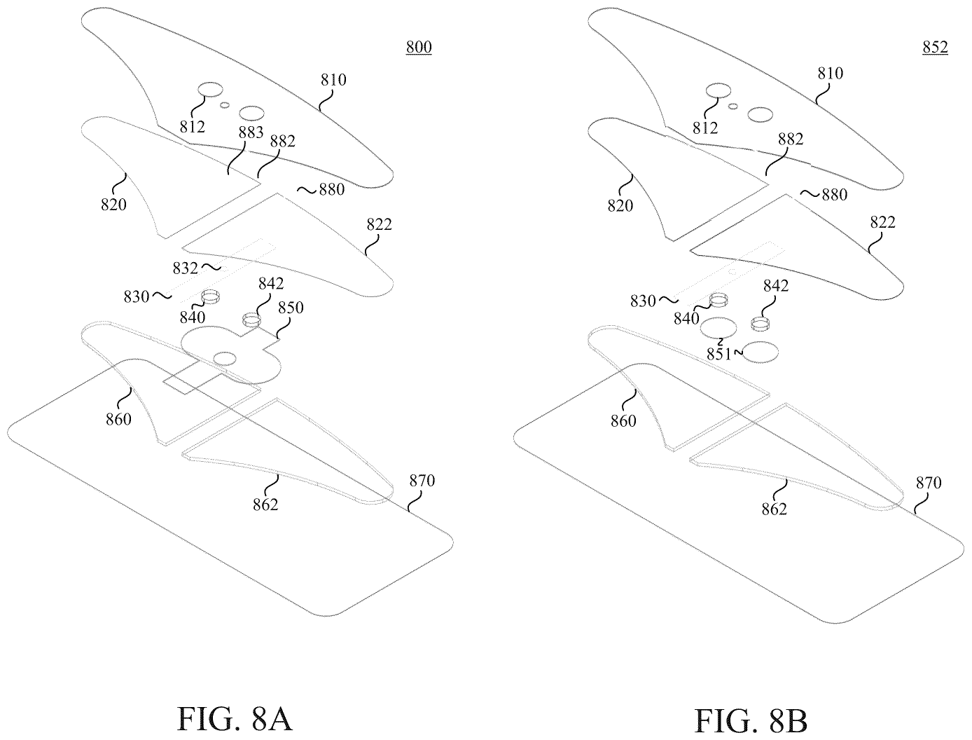

FIGS. 8A and 8B are respective schematic exploded perspective views of illustrative variations of an electrode 800, 852. As shown in in FIG. 8A, an electrode 800 may comprise a substrate 810 (e.g., backing material), a first conductor 820, a second conductor 822, an insulator 830 (e.g., non-woven fabric), a first connector 840, a second connector 842, connector substrate 850, a first adhesive conductor 860, a second adhesive conductor 862, a release liner 870, and an electrode identifier 880. FIG. 8B illustrates an electrode 852 similar to electrode 800 but having a circular connector substrate 851. In some variations, a plurality of connectors 840, 842 may be disposed on a single connector substrate 850 as shown in FIG. 8A while FIG. 8B depicts each connector 840, 842 disposed on respective connector substrates 851. The connector substrate 850 may comprise a shape that overlaps the space between the first and second adhesive conductors 860, 862 (e.g., space corresponding to an insulator). The connector substrate 850 may define an aperture for ground. In some variations, the connector substrate 850 may comprise a shape that overlaps a portion of respective first and second adhesive conductors 860, 862.

In some variations, the first and second connector 840, 842 may comprise a cylindrical body. In other variations, the set of connector 840, 842 may comprise other shapes (e.g., rectangular body, rounded, semi-spherical). As another example, one or more of the connector may comprise a curved shape (e.g., C-shaped).

In some variations, the first and second connector 840, 842 may have the same configuration (e.g., dimensions, shape). In other variations, the first and second connectors 840, 842 may have different configurations. In some variations, the connectors 840, 842 may have a thickness of between about 0.4 mm and about 1.0 mm, including all ranges and sub-values in-between. In some variations, the connectors 840, 842 may be metallic.

In some variations, the first conductor 820, the second conductor 822, and the insulator 830 may each be disposed on the substrate 810. The insulator 830 may be positioned laterally between the first and second conductors 820, 822. The first and second conductors 820, 822 may be configured to receive a set of pulses to stimulate a nerve of the patient. The electrode identifier 880 may be disposed on the substrate 810 and across (e.g., overlapping, over, intersecting) the first and second conductors 820, 822, and insulator 830.

In some variations, the electrode identifier 880 may comprise a set of apertures 882 (e.g., at least two apertures) and the substrate 810 may comprise a corresponding set of apertures 812. The first connector 840 may be coupled to the first adhesive conductor 860, and the second connector 842 may be coupled to the second adhesive conductor 862. The first and second connectors 840, 842 may be configured to be aligned with, and project through a respective aperture 882 of the electrode identifier 880 and a respective aperture 812 of substrate 810. The electrode identifier apertures 882 may also be aligned with, and may overlap, the substrate apertures 812.

In this manner, the first and second connectors 840, 842 may be configured to receive a set of pulses generated by a signal generator (e.g., signal generator 116). The set of apertures 882 may include a third aperture 883, and the insulator 830 may define a fourth aperture 832 corresponding (e.g., overlapping) the third aperture 883 of the insulator for ground.

As shown in FIGS. 8A and 8B, the electrode identifier 880 may overlap the first and second conductors 820, 822 in a lateral direction of the electrode 800, 852, and may be between the substrate 810 and the first and second conductors 820, 822 in a thickness direction of the electrode 800, 852 (e.g., from the front of the housing to the back of the housing). Furthermore, the electrode identifier 880 may be disposed on and overlap the insulator 830. In some variations, the electrode identifier 880 may comprise a Radio Frequency Identification (RFID) tag. While depicted as an RFID tag, the electrode identifier may be any of those described herein, such as, for example, a QR code, barcode, text, label, memory, and the like. This configuration may minimize interference due to the RFID tag without reducing electrode performance and/or substantially increasing the size of the electrode.

The dimensions described herein permit the electrodes to deliver a set of pulses to the nerves of the patient. In some variations, the magnets as described herein may be formed of any biocompatible conductive metal and/or alloy including, but not limited to tungsten, silver, platinum, platinum-iridium, nickel titanium alloys, copper-zinc-aluminum-nickel alloys, and copper-aluminum-nickel alloys, combinations thereof, and the like.

a. Electrode Identifier

Generally, an electrode as described herein may comprise an electrode identifier that may be detected by an electrode identifier reader and used to determine the suitability of the electrode for use with a stimulation system. For example, when the electrode has been identified as authentic and appropriately configured (e.g., properly coupled to a stimulation system) based on a detected electrode identifier, then a set of pulses may be generated and delivered to the electrode to thereby stimulate a nerve of a patient. Conversely, when the electrode has not been detected and/or an unauthorized, expired, and/or overused electrode has been identified, then generation of the set of pulses may be inhibited and/or a notification may be output (e.g., audio and/or visual warning). This may prevent damage to the patient and/or decrease the likelihood of sub-optimal treatment due to deteriorated electrode performance.

In some variations, an electrode configured to be coupled to a forehead of the patient may comprise a substrate, a first conductor, a second conductor, and an insulator each disposed on the substrate, the insulator positioned laterally between the first and second conductor, the first and second conductors configured to stimulate a trigeminal nerve of the patient. An electrode identifier may be disposed on the substrate and across the first and second conductors.

In some variations, the electrode may comprise an electrode identifier such as a radiofrequency identification (RFID) tag, QR code, barcode, text, label, memory, and the like. For example, the electrode identifier may be printed on a surface (e.g., surface of a substrate, surface of a conductor), adhered to the surface by an adhesive, combinations thereof, and the like. In some variations, the electrode identifier may be configured to electronically store data such as a unique identifier for the electrode. The electrode identifier may be used to track the electrode and/or may be used to authenticate the electrode. In some variations, the electrode identifier may be configured to wirelessly transmit data (e.g., authentication information, usage information) to an identifier reader of a transcutaneous electrical stimulation system. For example, in variations in which the electrode identifier may comprise an RFID tag, the RFID tag may comprise data such as a unique identifier and a password for the electrode.

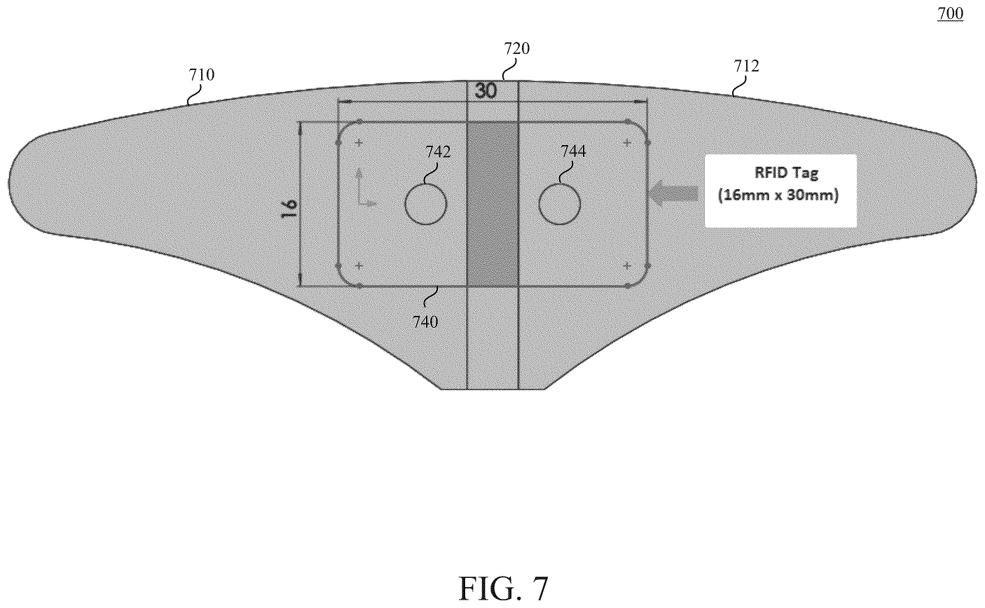

Turning to FIG. 7, depicted there is a schematic plan view of an illustrative variation of an electrode 700 including a first conductor 710, a second conductor 712, an insulator 720, and an electrode identifier 740. The electrode identifier 740 may be disposed on (e.g., coupled to) and positioned laterally across (e.g., overlapping) the first conductor 710, the second conductor 712, and the insulator 720. In some variations, the electrode identifier 740 may comprise a length of about 30 mm and a width of about 16 mm. In some variations, the electrode identifier 740 may be centered along one or more of a vertical and horizontal axis of the electrode.

The electrode identifier 740 may comprise apertures 740, 742 configured to receive respective connectors (e.g., magnets) that may project (e.g., protrude) through the apertures 740, 742 of the electrode identifier 740 to couple to the system housing and/or components contained there (durable components). This configuration may reduce the size and/or weight of the electrode without reducing electrode performance, which may improve the ergonomics of having an electrode attached to a patient's body (e.g., the forehead). Alignment of the electrode identifier 740 with each of the conductors 710, 712, insulator 720, and magnets may facilitate a compact electrode and may reduce power requirements for reading the electrode identifier 740.

b. Connector

Generally, a stimulation system (e.g., wearable system 100) may comprise a connector (e.g., connector 132) configured to releasably couple a housing of the system (and/or components contained therein) to an electrode. The connector may be configured to mechanically and/or magnetically couple the electrode to the system. In this manner, the electrode may be used as a disposable component having a predetermined number of uses while the housing of the stimulation system and the components contained therein may be a durable component that may be re-used as desired.

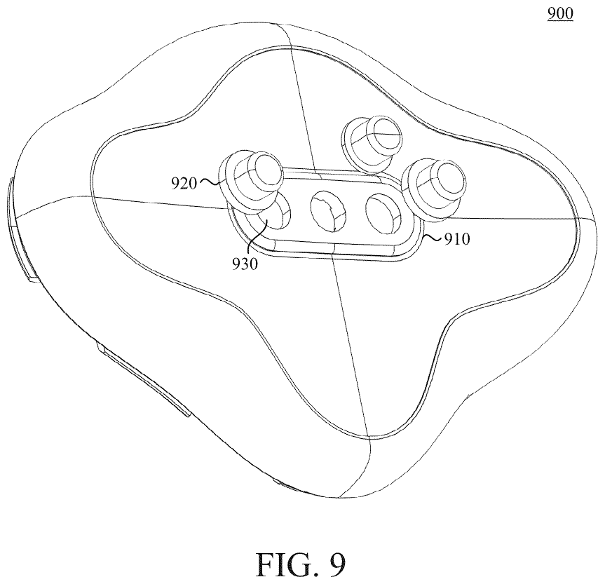

FIG. 9 is a rear exploded perspective view of an illustrative variation of a connector of a transcutaneous electrical stimulation system 900. In some variations, the connector may comprise a connector body 910 and a set of magnets 920 configured to magnetically couple to corresponding connectors on an electrode (e.g., connectors 620). The magnets 910 may be coupled to one or more lead wires (not shown) that may be coupled to a signal generator that is configured to generate a set of pulses. The magnets 910 may be configured to extend partially into respective apertures 930 of the connector body 910. In some variations, the connector body 910 may comprise a set of recesses configured to receive the corresponding magnets on the electrode. In some variations, the connector body 910 may protrude from a housing of the system 900. The connector body 910 may comprise rounded surfaces so as to be atraumatic. In some variations, the connector may be centered over one or more of a horizontal axis and vertical axis of the system 900. In some variations, the set of magnets 920 may be circular or any of the shapes as discussed herein.

In some variations, a diameter or width of each of the apertures 930 may be between about 3 mm and about 5 mm. In some variations, a center-to-center distance between the outermost apertures 930 may be between about 10 mm and about 15 mm.

In some variations, a magnet 920 may be coupled to one or more electrically conductive wires (e.g., lead wire) configured to connect the electrode to one or more components of the durable component including a signal generator, processor, and the like. In some variations, each magnet may be coupled to a respective insulated lead wire formed of any electrically conductive metal and/or biocompatible conductive metal and/or alloy including but not limited to copper, silver, platinum, platinum-iridium, combinations thereof, and the like. One or more portions of the lead wires may be flexible or semi-flexible, one or more portions may be rigid or semi-rigid, and/or one or more portions of the lead wires may transition between flexible and rigid configurations. The lead wires described herein may be made of any material or combination of materials. For example, the lead wires may be insulated using one or more polymers (e.g., silicone, polyvinyl chloride, latex, polyurethane, polyethylene, PTFE, nylon).

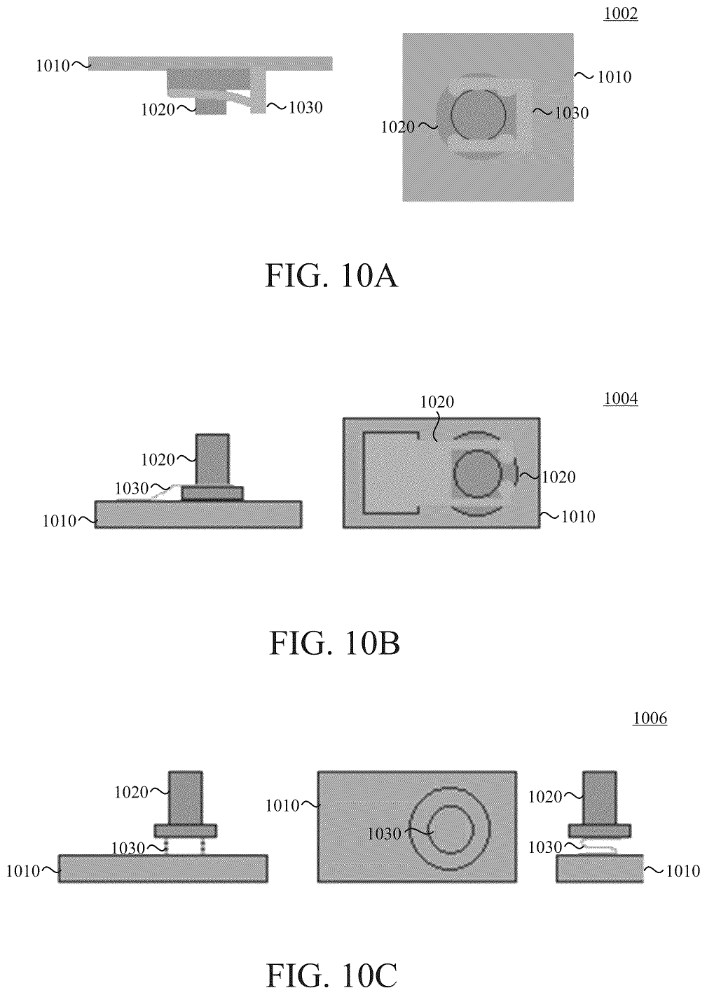

In some variations, the magnets of a connector may be coupled to a substrate such as an electronic circuit (e.g., electronic circuit 460) such that the magnets may protrude through an aperture of a system housing and couple to an electrode while being fixed to the substrate. FIGS. are schematic side views of illustrative variations of magnets 1020 of transcutaneous electrical stimulation systems 1002, 1004, 1006 coupled to an electronic circuit 1010. In some variations, a fastener 1030 may be configured to hold the magnet 1020 onto the electronic circuit 1010 to maintain the coupling between the magnet 1020 and the electronic circuit 1010 as shown in FIGS. 10A-10C. In FIG. 10A, the fastener 1030 may be configured to apply a pushing force towards the electronic circuit 460. For example, the fastener 1030 may comprise a spring configured to hold the fastener 1030 to the surface of the electronic circuit 460. In FIG. 10C, a spring fastener 1030 may be coupled between the magnet 1020 and the electronic circuit 1010.

Additionally or alternatively, an electronic circuit (e.g., flexible printed circuit board (PCB)) may comprise a set of recesses each sized to receive and hold a corresponding magnet. In some variations, a magnet may be coupled (e.g., using an adhesive) to a rigid surface of a PCB (e.g., rigid-flex-rigid PCB).

B. Signal Generator

Generally, a signal generator of any of the systems, devices, and methods described herein may be configured to generate a set of pulses for transcutaneous stimulation of a nerve of a patient. In some variations, a signal generator may comprise a high voltage generator and a current pulse generator. The high voltage generator may be configured to convert power (e.g., up to 100 V) from a power source (e.g., battery) to a high voltage signal which may be input to a current pulse generator. The current pulse generator may be configured to convert the high voltage signal to a set of pulses of a treatment stimulation program having a predetermined set of parameters (e.g., duration, intensity) as described in more detail herein. In some variations, the current pulse generator may comprise at least two transistors.

In some variations, a signal generator may be configured to generate a set of pulses based on a set of parameters comprising one or more of a pulse frequency, a pulse width, a pulse period, a pulse amplitude, a ramp up time, a steady time, a ramp down time, a session duration, a phase charge, a rise time, a dead period, and an overshoot.

In some variations, a phase charge may be up to about 5 μC. In some variations, a rise time of a pulse may be up to about 5 μs at about 50% of the maximum.

In some variations, the signal generator may be configured to generate the set of pulses comprising a pulse width between about 240 μs and about 260 μs, a pulse amplitude of up to about 17 mA, and a dead time of between about 1 μs and about 10 μs.

In some variations, the signal generator may be configured to generate a different set of pulses (e.g., pulses with different parameters, treatment stimulation program) based on one or more of the treatment indication and/or treatment type (e.g., acute, preventative). For example, a set of pulses configured to treat an acute migraine may comprise a pulse frequency of about 100 Hz, a pulse width of about 250 μs, a pulse period of about 500 μs, a maximum pulse amplitude of about 16 mA, a ramp up time of about 14 minutes, a steady time of about 46 minutes, a ramp down time of about 45 seconds, and a session duration of about 60 minutes.

In some variations, a set of pulses configured to prevent a migraine may comprise a pulse frequency of about 60 Hz, a pulse width of about 250 μs, a pulse period of about 500 μs, a maximum pulse amplitude of about 16 mA, a ramp up time of about 14 minutes, a steady time of about 6 minutes, a ramp down time of about 45 seconds, and a session duration of about 20 minutes.

In some variations, the signal generator may incrementally increase the pulse amplitude, which may, for example, assist in reducing side effects. For example, a treatment stimulation program may comprise the following sequence of: increase a pulse amplitude from 0 mA to a first amplitude for a first duration; remain at the first amplitude for a second duration; increase the pulse amplitude from the first amplitude to a second amplitude at a first rate; remain at the second amplitude for a third duration; reduce the pulse amplitude from the second amplitude to a third amplitude at a second rate; remain at the third amplitude for a fourth duration; reduce the pulse amplitude from the third amplitude to a fourth amplitude at a third rate; remain at the fourth amplitude for a fifth duration; reduce from the fourth amplitude to a fifth amplitude at a fourth rate; remain at the fifth amplitude for a sixth duration; reduce from the fifth amplitude to a sixth amplitude at a fifth rate; remain at the sixth amplitude for a seventh duration; reduce from the sixth amplitude to a seventh amplitude at a sixth rate; remain at the seventh amplitude for an eighth duration; reduce from the seventh amplitude to an eight amplitude for a ninth duration.

In some variations, the signal generator may incrementally increase the pulse amplitude, which may, for example, assist in reducing side effects. For example, a treatment stimulation program may comprise the following sequence of: increase a pulse amplitude from 0 mA to about 1.5 mA in about 10 ms; remain at about 1.5 mA for about 250 ms; increase the pulse amplitude from about 1.5 mA to about 25 mA by steps of about 0.5 mA each 30 ms; remain at about 25 mA for about 1 second; reduce the pulse amplitude from about 25 mA to about 20 mA in about 50 ms; remain at about 20 mA for about 1 second; reduce the pulse amplitude from about 20 mA to about mA in about 50 ms; remain at about 15 mA for about 1 second; reduce from about 15 mA to about 10 mA in about 50 ms; remain at about 10 mA for about 1 second; reduce from about 10 mA to about 5 mA in about 50 ms; remain at 5 mA for about 1 second; reduce from about 5 ma to about 1.5 mA in about 50 ms; remain at about 1.5 mA for about 1 second; reduce from about 1.5 mA to 0 mA in about 50 ms.

In some variations, the signal generator may incrementally increase the pulse amplitude, which may, for example, assist in reducing side effects. For example, a treatment stimulation program may comprise the following sequence of: increase a pulse amplitude from 0 mA to about mA in about 10 ms; remain at about 0.32 mA for about 250 ms; increase the pulse amplitude from about 0.32 to about 25 mA by steps of about 0.5 mA each 30 ms; remain at about 25 mA for about 1 second; reduce the pulse amplitude from about 25 mA to about 20 mA in about 50 ms; remain at about 20 mA for about 1 second; reduce the pulse amplitude from about 20 mA to about mA in about 50 ms; remain at about 15 mA for about 1 second; reduce from about 15 mA to about 10 mA in about 50 ms; remain at about 10 mA for about 1 second; reduce from about 10 mA to about 5 mA in about 50 ms; remain at 5 mA for about 1 second; reduce from about 5 ma to about 1.5 mA in about 50 ms; remain at about 1.5 mA for about 1 second; reduce from about 1.5 mA to 0 mA in about 50 ms.

C. Electrode Identifier Reader

Generally, an electrode identifier reader (e.g., RFID reader) may be configured to communicate with the electrode identifier to receive data corresponding to the electrode. For example, the electrode identifier reader may be configured to receive one or more of electrode data, usage data, and authentication data.

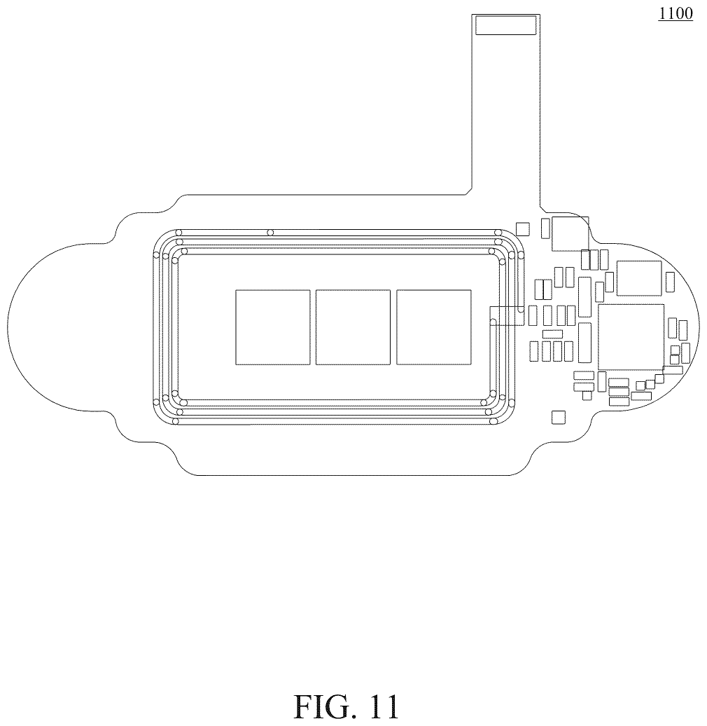

In some variations, the electrode identifier and the electrode identifier reader may be placed within a predetermined proximity of each other (e.g., disposable electrode coupled to a durable system housing) to facilitate communication and/or data transfer. For example, coupling between the stimulation system and electrode may be determined based on a measured load. If a predetermined load (e.g., electrodes) is detected, then the electrode may be authenticated using the electrode identifier reader and electrode identifier. In some variations, the electrode identifier reader may comprise one or more of an RFID reader, a barcode reader, an optical character reader, and the like. For example, FIG. 11 is a schematic plan view of an illustrative variation of an RFID circuit 1100 of a transcutaneous electrical stimulation system. In some variations, as shown in FIG. 4, the electrode identifier reader 480 may be disposed between a rear housing 440 and the power source 470. That is, the electrode identifier reader 480 is disposed so as to be as close as practical to the rear housing 440 in order to increase sensitivity and reduce power consumption of the electrode coupled to the system 400. In some variations, the electrode identifier reader may comprise an antenna configured to generate a magnetic field at a predetermined frequency (e.g., about 13 MHz).

Additionally or alternatively, the electrode identifier reader may comprise an optical sensor (e.g., CCD, camera) configured to generate an image of the electrode identifier (e.g., QR code).

D. Input Device

Generally, an input device of a transcutaneous electrical stimulation system may serve as a control interface for a patient. In some variations, the system may comprise one or more input devices. For example, the wearable system 100 may comprise an input device 120 (e.g., button switch) configured to control the wearable system 100. Additionally or alternatively, the compute device 160 may comprise a corresponding input device (e.g., touchscreen interface) configured to control the wearable system 100. In some variations, the input device 120 may be configured to receive input to control one or more of the signal generator 116, the output device 122, the communication device 128, and the like. For example, patient actuation of an input device 120 (e.g., switch 310, 410, 420, 1200) may be processed by the processor 124 and the memory 126 to output a control signal to signal generator 116.

Some variations of an input device may comprise at least one switch configured to generate a control signal. In some variations, the input device may encompass at least about 50% of a first side (e.g., front facing) of the system housing, thereby providing a larger surface area for contact. For example, FIGS. 3A and 3C depict a switch 310 that covers at least about 50% of a surface area of a first side of the system 300. In some variations, a switch may be configured to cover at least about 60%, at least about 70%, at least about 80%, at least about 90%, including all ranges and sub-values in-between. When the system is coupled to a forehead of the patient, the input device cannot be seen by the patient such that a larger contact area may facilitate non-visual operation of the system. In some variations, the switch 310 may comprise a single button. In some variations, the switch may comprise a plurality of buttons (e.g., actuator) located at different portions of the housing. For example, one or more portions of the switch 310 (e.g., upper portion, lower portion, first lateral portion, second lateral portion) may correspond to a respective button configured for a respective set of functions.

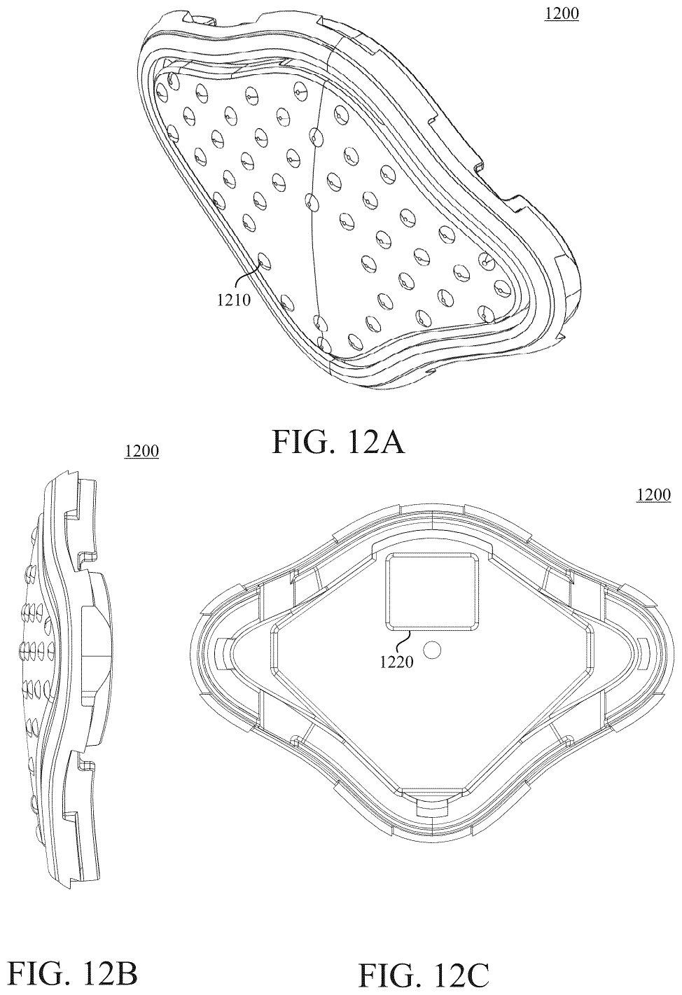

In some variations, the input device of a transcutaneous electrical stimulation system may include a switch cover 410 and a switch 420, as shown in FIG. 4. The switch cover 410 may comprise a non-deformable material such as the same or a similar material as the system housing and the switch 420 may comprise a resilient, deformable material such that the switch 420 functions as a button. Similarly, FIGS. 12A-12C are respective front perspective, side, and rear views of a switch 1200 of a transcutaneous electrical stimulation system. In some variations, the switch 420, 1200 may comprise a deformable material (e.g., rubber, silicone) configured for repeat tactile actuation by a patient. In some variations, the switch 410, 1200 may comprise a set of protrusions 1210 facing a first side of the housing. In some variations, adjacent protrusions 1210 may be separated by about 5 mm. The set of protrusions 1210 may be configured to face and/or contact a switch cover (not shown in FIGS. 12-12C). The switch 410, 1200 may comprise a recess 1220 on a second side of the housing opposite the first side of the housing. One or more components of the system (e.g., processor, memory of electronic circuit 460) may be disposed within the recess 1220. For example, the recess 1220 may have a length of about 12 mm and a width of about 10 mm.

Additionally or alternatively, in variations of an input device comprising at least one switch, a switch may comprise, for example, at least one of a button (e.g., hard key, soft key), touch surface, keyboard, analog stick (e.g., joystick), directional pad, mouse, trackball, jog dial, step switch, rocker switch, pointer device (e.g., stylus), motion sensor, image sensor, and microphone. A motion sensor may receive a signal from an optical sensor and classify a patient gesture as a control signal. A microphone may be configured to receive audio and recognize a voice (e.g., verbal command) as a control signal. In variations of a system comprising a plurality of input devices, different input devices may generate different types of signals. For example, some input devices (e.g., button on stimulation system) may be configured to generate a control signal to start/stop treatment while other input devices (e.g., touchscreen of compute device) may be configured to generate a control signal to modify stimulation parameters (e.g., time, intensity).

In variations of the input device comprising one or more buttons, button presses of varying duration may execute different functions. For example, a longer button press may correspond to selecting a preventative treatment stimulation program. Conversely, a shorter duration button press may, for example, correspond to selecting an acute treatment stimulation program. As another example, a first button (e.g., located at a top portion of the switch) may correspond to a first set of functions including selecting a stimulation treatment program (e.g., acute treatment, preventative treatment), a second button (e.g., located at a bottom portion of the switch) may correspond to a second set of functions including changing one or more of an intensity (e.g., amplitude) and duration of the treatment, and a third button (e.g., located at a lateral portion of the switch) may correspond to a third set of functions including connecting the stimulation system to a mobile application of a compute device (e.g., mobile app running on a mobile phone).

E. Output Device

Generally, an output device 130 of a transcutaneous electrical stimulation wearable system 100 and/or compute device 160, 162 may be configured to output data corresponding to a transcutaneous electrical stimulation system, and may comprise one or more of a display device (e.g., set of LEDs), audio device (e.g., buzzer), and haptic device. In some variations, an output device may comprise a display device including at least one of a light emitting diode (LED), liquid crystal display (LCD), electroluminescent display (ELD), plasma display panel (PDP), thin film transistor (TFT), organic light emitting diodes (OLED), electronic paper/e-ink display, laser display, and/or holographic display.

In some variations, the display device 130 may comprise one or more LEDs (e.g., one, two, three, four, or more) and may include a tricolor LED (e.g. red, green, blue). In some variations, the output device 130 may be configured to indicate, for example, a status of the device. For example, the output device 130 may be configured to indicate one or more of a treatment program (e.g., acute treatment, prevent treatment), a sleep state, a standby state, a battery charge state (e.g., low, charged, charging, voltage value), a compute device connection state, and an electrode authentication state. In some variations, the display device 130 may comprise one or more portions of the switch cover 410 and the switch 420 having a transparent portion and/or translucent portion configured to output light generated by the set of LEDs. Put another way, the display device 130 may comprise a portion of the switch cover 410 and/or switch 420 that is transparent and/or translucent.

In some variations, the output device 130 may comprise an optical waveguide (e.g., light pipe, light distribution guide, etc.). One or more optical waveguides may receive light from a light source (e.g., illumination source) using a predetermined combination of light output parameters (e.g., wavelength, frequency, intensity, pattern, duration). In some variations, the optical waveguide may be formed integrally with one or more portions of the housing (e.g., switch cover) of the device. An optical waveguide may refer to a physical structure that guides electromagnetic waves such as visible light spectrum waves to passively propagate and distribute received electromagnetic waves. Non-limiting examples of optical waveguides include optical fiber, rectangular waveguides, light tubes, light pipes, combinations thereof, or the like. For example, light pipes may comprise hollow structures with a reflective lining or transparent solids configured to propagate light through total internal reflection. The optical waveguides described herein may be made of any suitable material or combination of materials. For example, in some variations, the optical waveguide may be made from optical-grade polycarbonate. In some variations, the housings as described herein may be co-injected molded to form the optical waveguides. In other variations, the optical waveguides may be formed separately and coupled to the housing. In some variations, the optical waveguides described herein may comprise one or more portions configured to emit light. For example, at least one of the portions may comprise one or more shapes. For example, the optical waveguide may follow the edges of the housing and/or form a shape of a logo. In some variations, the optical waveguides described herein may comprise a surface contour including, for example, a multi-faceted surface configured to increase visibility from predetermined vantage points.

The light patterns described herein may, for example, comprise one or more of flashing light, occulting light, isophase light, etc., and/or light of any suitable light/dark pattern. For example, flashing light may correspond to rhythmic light in which a total duration of the light in each period is shorter than the total duration of darkness and in which the flashes of light are of equal duration. Occulting light may correspond to rhythmic light in which the duration of light in each period is longer than the total duration of darkness. Isophase light may correspond to light which has dark and light periods of equal length. Light pulse patterns may include one or more colors (e.g., different color output per pulse), light intensities, and frequencies.

In some variations, the transcutaneous electrical stimulation system may additionally or alternatively comprise an output device such as an audio device and/or a haptic device. For example, an audio device may audibly output patient data, stimulation data (e.g., treatment program), error data, system data (e.g., power source status), authentication data (e.g., electrode authentication), alarms, and/or notifications (e.g., treatment started, treatment ended). For example, the audio device may output an audible alarm when a power source has insufficient power or when an unauthorized electrode is coupled to the signal generator of the device. In some variations, an audio device may comprise at least one of a speaker, a piezoelectric audio device, a magnetostrictive speaker, and/or digital speaker. In some variations, a patient may communicate with other users using the audio device (e.g., microphone) and a communication channel. For example, a user may form an audio communication channel (e.g., cellular call, VoIP call) with a health care provider or another person.

In some variations, an audio device of output device 130 and/or compute device 160, 162 may be configured to indicate a status of the device. For example, the audio device (and any of the output devices described herein) may be configured to indicate a power state (e.g., ON), a treatment program (e.g., acute treatment, prevent treatment), a current amplitude (e.g., steady state amplitude, maximum current reached, current manually increased), a sleep state, a standby state, a battery charge state (e.g., low, charged, charging, voltage value), a compute device connection state, a patient connection state (e.g., electrode coupled to a forehead of the patient), and an electrode authentication state.

In some variations, a haptic device may be incorporated into the transcutaneous electrical stimulation system and/or compute device 160, 162 to provide additional sensory output (e.g., force feedback) to the patient. For example, a haptic device may generate a tactile response (e.g., vibration) to confirm user input to an input device (e.g., button) or to communicate an operation state (e.g., first vibration pattern corresponding to a first operation state, second vibration pattern corresponding to a second operation state).

F. Housing

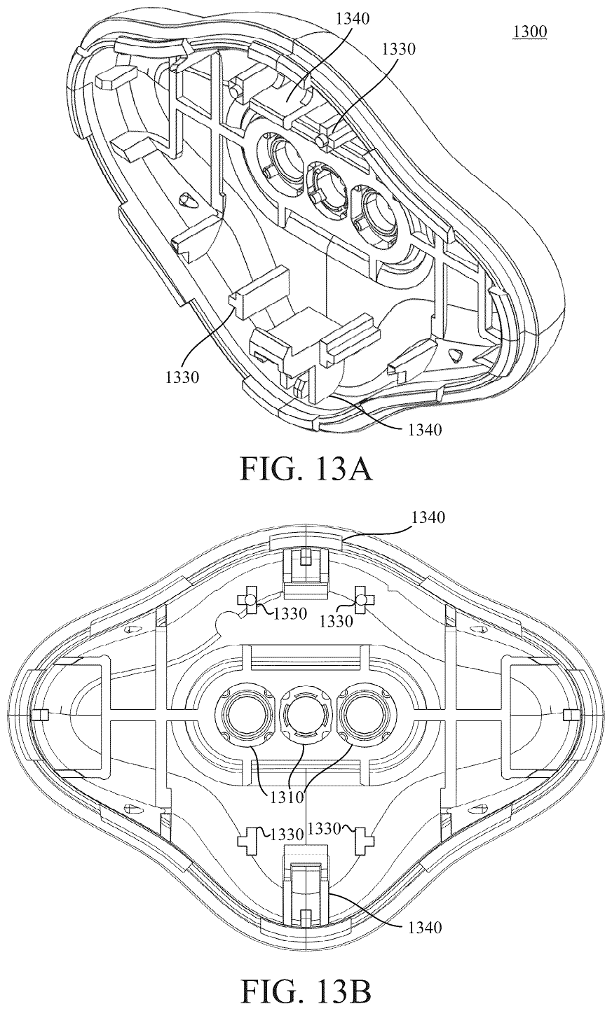

Generally, a housing of a transcutaneous electrical stimulation system may be configured to enclose a set of durable elements in a compact and lightweight form factor that may be held to a skin surface of a patient (e.g., forehead) while the patient receives treatment. Furthermore, the housing of the system may be configured to arrange the components disposed therein to improve performance of the system. As described with respect to FIG. 4, the electronic circuit 460, power source 470 and spacer 490, and electrode identifier reader 480 may be sequentially arranged within the housing. FIGS. 13A and 13B are respective front perspective and front views of a rear housing of a transcutaneous electrical stimulation system configured to secure the components of the stimulation system in place relative to each other.

In some variations, the housing of the stimulation device may be configured to separate the power source from the signal generator (e.g., maintain a predetermined distance between the electronic circuit and the power source). For example, a set of protrusions 1330 and a set of fasteners 1340 (e.g., hooks) of a rear housing may be configured to couple to an electronic circuit (e.g., including a signal generator) such that the electronic circuit is held in place within the housing 1300 of the system. For example, the set of protrusions 1330 and fasteners 1340 may be configured to couple (e.g., attach) to the electronic circuit such that an electronic circuit 460 is separated from a power source 470. The spacing created by the protrusions 1330 and fasteners 1340 may accommodate heat and dimensional changes in the power source 470 (e.g., due to battery swelling), thereby improving system performance and extending a lifespan of the system. The set of fasteners 1340 (e.g., hooks, clips) may further be configured to couple (e.g., attach) the electronic circuit 460 (e.g., an edge of the electronic circuit) to the housing 1300. In some variations, a distance between fasteners (e.g., upper fastener, lower fastener) may be between about 25 mm and about 35 mm. In some variations, the protrusions 1330 may comprise a height between about 5 mm and about 10 mm, including all ranges and sub-values in-between. In some variations, the fasteners 1340 may comprise a height of between about 5 mm and about 10 mm, including all ranges and sub-values in-between. In some variations, the protrusions 1330 may comprise a first height and the fasteners 1340 may comprise a second height greater than the first height. For example, the first height may be about 7 mm and the second height may be about 9 mm. In some variations, the protrusions 1330 and fasteners 1340 may be disposed along opposing lateral ends of the housing. In some variations, an electronic circuit (e.g., electronic circuit 460) may be separated from a power source (e.g., power source 470) by up to about 2.0 mm, and up to about 1.0 mm, including all ranges and sub-values in-between.

In some variations, the set of protrusions 1330 may be configured to couple to a first side of the electronic circuit (e.g., signal generator) and the set of fasteners 1340 may be configured to couple to a second side of the electronic circuit opposite the first side. In some variations, the set of protrusions 1330 may be configured to separate the electronic circuit (e.g., signal generator) from the connector 1310 by a second predetermined distance. For example, the electronic circuit may be separated from the housing by up to about 10 mm, up to about 7 mm, up to about 5 mm, including all ranges and sub-values in-between. In some variations, a height of the set of protrusions 1330 may be less than a height of the set of fasteners 1340. In some variations, the set of protrusions 1330 and the set of fasteners 1340 may be located on opposite ends of the housing (e.g., top and bottom, left and right) such that the set of protrusions 1330 and fasteners 1340 do not contact the power source. FIGS. 13A and 13B illustrate a set of four protrusions 1330, but the set of protrusions 1330 may include any number of protrusions including 1, 2, 3, 4, 5, 6, 7, 8, 9, 10, or more. Similarly, FIGS. 13A and 13B illustrate a set of two fasteners 1340, but the set of fasteners 1340 may include any number of fasteners including 1, 2, 3, 4, 5, 6, 7, 8, 9, 10, or more. In some variations, the set of protrusions 1330 may be configured to contact the electronic circuit and/or protrude through a corresponding aperture in the electronic circuit to hold the electronic circuit in place relative to the housing.

G. Processor

A transcutaneous electrical stimulation wearable system 100, as depicted in FIG. 1, may comprise a processor 124 and a machine-readable memory 126 (e.g., collectively a controller) in communication with one or more compute devices 160, 162. The processor 124 may be connected to the compute devices 160, 162 by wired or wireless communication channels. The processor 124 may be configured to control one or more components of the wearable system 100, such as the signal generator 116, the electrode identifier reader 118, and the communication device 128. The processor 124 may be implemented consistent with numerous general purpose or special purpose computing systems or configurations. Various exemplary computing systems, environments, and/or configurations that may be suitable for use with the systems and devices disclosed herein may include, but are not limited to software or other components within or embodied on personal computing devices, network appliances, servers or server computing devices such as routing/connectivity components, portable (e.g., hand-held) or laptop devices, multiprocessor systems, microprocessor-based systems, and distributed computing networks.

The processor 124 may incorporate data received from the memory 126, patient input, and compute device(s) 160, 162 to control the system(s) 10, 100. The memory 126 may further store instructions to cause the processor 124 to execute modules, processes, and/or functions associated with the system 100 and/or compute device(s) 160, 162. The processor 124 may be any suitable processing device configured to run and/or execute a set of instructions or code and may comprise one or more microcontrollers, data processors, image processors, graphics processing units, physics processing units, digital signal processors, and/or central processing units. The processor 124 may be, for example, a general purpose processor, a Field Programmable Gate Array (FPGA), an Application Specific Integrated Circuit (ASIC), configured to execute application processes and/or other modules, processes, and/or functions associated with the system and/or a network associated therewith. For example, the processor 124 may be a dual core microcontroller. The underlying device technologies may be provided in a variety of component types such as metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, combinations thereof, and the like.

H. Memory

Some variations of memory 126 described herein relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as air or a cable). The media and computer code (also may be referred to as code or algorithm) may be those designed and constructed for a specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical discs; solid state storage devices such as a solid state drive (SSD) and a solid state hybrid drive (SSHD); carrier wave signal processing modules; and hardware devices that are specially configured to store and execute program code such as Application-Specific Integrated Circuits (ASICs), Programmable Logic Devices (PLDs), Read-Only Memory (ROM), and Random-Access Memory (RAM) devices. Other variations described herein relate to a computer program product, which may include, for example, the instructions and/or computer code disclosed herein.

The systems, devices, and/or methods described herein may be performed by software (executed on hardware), hardware, or a combination thereof. Software modules (executed on hardware) may be expressed in a variety of software languages (e.g., computer code), including C, C++, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

In some variations, a set of parameters may be stored in memory 126 and/or transmitted to a compute device 160 including a session time (e.g., treatment session timestamp or the time when a session is started), a treatment stimulation program selected, a session duration (e.g., number of minutes of the session), a maximum current amplitude in a session, a session error, a number of repetitions, a sum of current delivered, a sum of current delivered if maximum current amplitude was reached, a set of pulse parameters, a battery charge time (e.g., timestamp), a battery charge duration, a duration to reach full charge, and a battery charge error.

I. Communication Device

In some variations, transcutaneous electrical stimulation systems 100 described herein may communicate with networks and computer systems through a communication device 128. In some variations, the transcutaneous electrical stimulation wearable system 100 may be in communication with other devices (e.g., compute devices) via one or more wired and/or wireless networks. A wireless network may refer to any type of digital network that is not connected by cables of any kind. Examples of wireless communication in a wireless network include, but are not limited to Bluetooth, cellular, radio, satellite, and microwave communication. However, a wireless network may connect to a wired network in order to interface with the Internet, other carrier voice and data networks, business networks, and personal networks. A wired network is typically carried over copper twisted pair, coaxial cable and/or fiber optic cables. There are many different types of wired networks including wide area networks (WAN), metropolitan area networks (MAN), local area networks (LAN), Internet area networks (IAN), campus area networks (CAN), global area networks (GAN), like the Internet, and virtual private networks (VPN). Hereinafter, network refers to any combination of wireless, wired, public and private data networks that are typically interconnected through the Internet, to provide a unified networking and information access system.

In some variations, communication using the communication device 128 may be encrypted. Any of the data stored in memory 126 (e.g., set of parameters described herein) may be transmitted using the communication device 128.

Cellular communication may encompass technologies such as GSM, PCS, CDMA or GPRS, W-CDMA, EDGE or CDMA2000, LTE, WiMAX, and 5G networking standards. Some wireless network deployments combine networks from multiple cellular networks or use a mix of cellular, Wi-Fi, and satellite communication. In some variations, the network interface 116 may comprise a radiofrequency receiver, transmitter, and/or optical (e.g., infrared) receiver and transmitter. The communication device 128 may communicate by wires and/or wirelessly with one or more components of the system(s) 10, 100.

J. Power Source

Generally, the transcutaneous electrical stimulation systems described herein may receive power from an internal power source (e.g., lithium battery, disposable battery) and may be recharged using an external power source (e.g., wireless charger, wall outlet). The transcutaneous electrical stimulation system may receive power via a wired connection, and/or a wireless connection (e.g., induction, RF coupling, etc.). The transcutaneous electrical stimulation system may comprise one or more power algorithms configured to conserve energy and increase a lifespan of the transcutaneous electrical stimulation system.

K. Charger

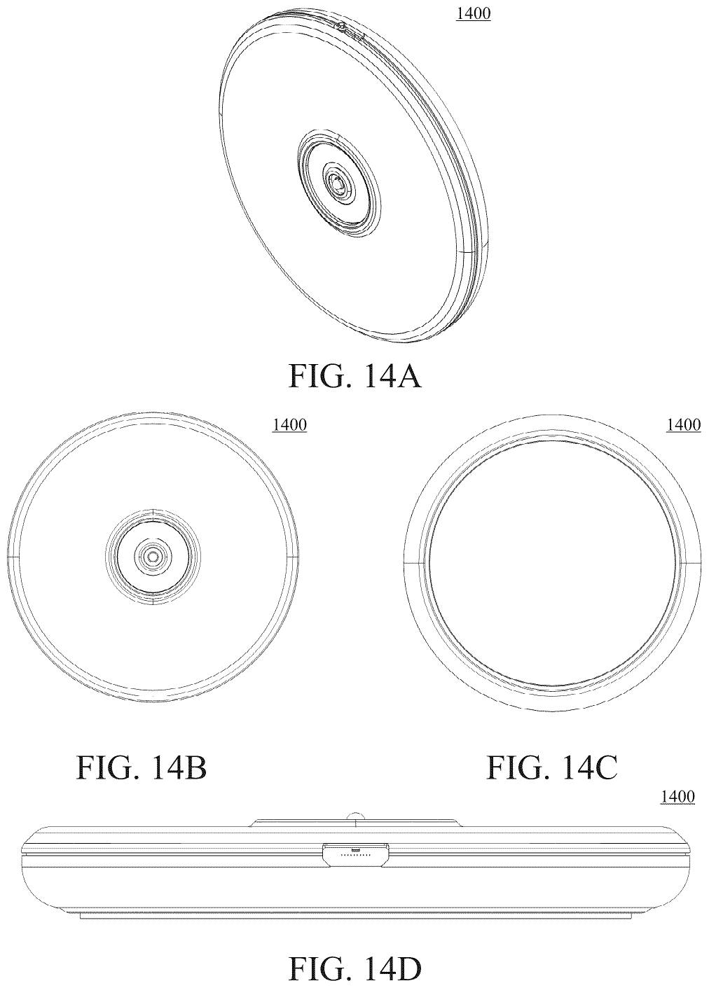

Generally, a charger may be configured to power and/or recharge a power source of a transcutaneous electrical stimulation system. FIGS. 14A-14D are respective front perspective, front, rear, and side views of a charger 1400. As shown in FIGS. 14A-14D, the charger 1400 may have a generally disk-like shape. In some variations, the charger 1400 may be configured to receive and hold a stimulation system. For example, the charger 1400 may comprise one or more recesses and/or protrusions to receive a rear portion of the stimulation system. In some variations, the charger 1400 may be electrically coupled to a power source of the stimulation system. In some variations, a system such as system 300 may be disposed on a surface of the charger 1400 to wirelessly transfer energy through inductive coupling (e.g., electromagnetic coupling) to the system 300. In some variations, the charger may comprise a USB-C cable.

II. Methods

Also described here are methods for non-invasive transcutaneous electrical stimulation using the devices and systems described herein. Generally, the methods described here comprise coupling an electrode to a skin surface (e.g., forehead) of the patient and coupling a housing of an electrical stimulation system to the electrode. In some variations, the electrode may be coupled to the housing of the electrical stimulation system prior to coupling the electrode to the skin surface, while in other variations, the electrode may be coupled to the skin surface, after which the housing of the electrical stimulation system may be coupled to the electrode while the electrode remains coupled to the skin surface (i.e., in-situ). In some variations, one or more of the stimulation system and/or compute device may provide instructions on how to couple the electrode and/or electrode to the patient, and/or how to use the system (e.g., select and initiate a treatment session). The electrode may comprise an electrode identifier for the electrode and the electrical stimulation system may comprise a signal generator and an identifier reader. The electrode identifier may be detected using the identifier reader of the electrical stimulation system. An authentication signal may be generated based on the detected identifier, and a nerve (e.g., trigeminal nerve) of the patient may be stimulated using a set of pulses generated by the signal generator based on the authentication signal. In some variations, methods may further comprise releasing the system from the electrode and/or removing the electrode from the skin surface, which may occur simultaneously or sequentially.

A. Applying Electrical Stimulation

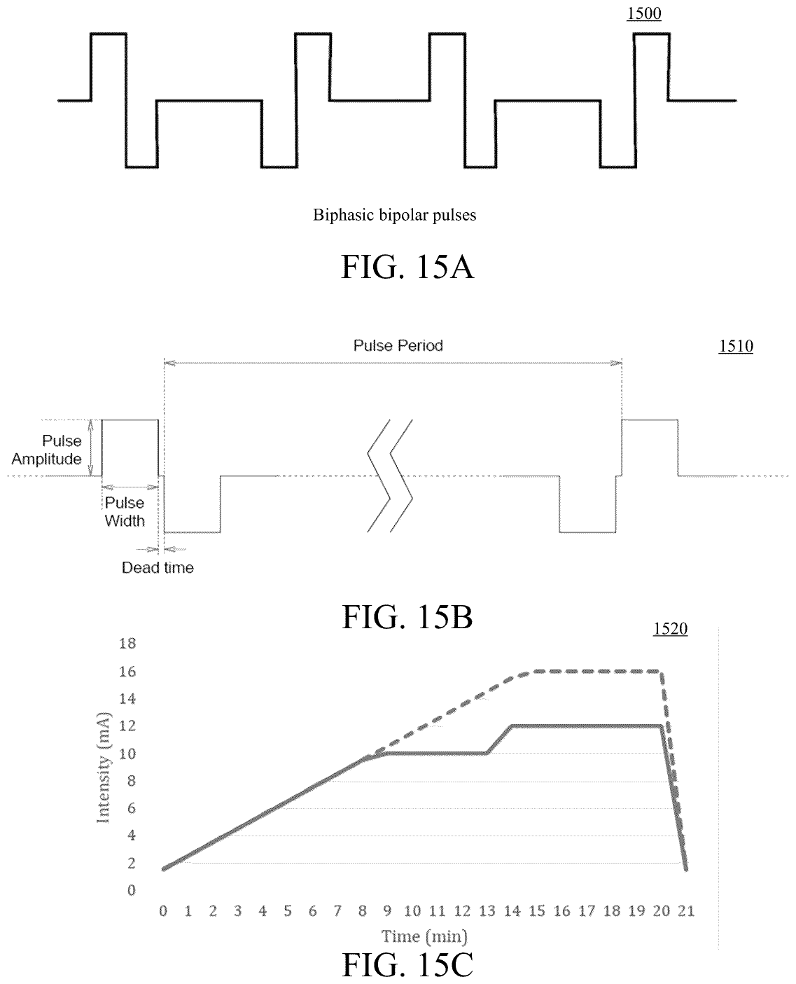

FIGS. 15A and 15B are plots of illustrative variations of pulse waveforms for transcutaneous electrical stimulation. For example, FIGS. 15A and 15B illustrate a set of biphasic bipolar pulses (e.g., stimulation signal) having an excitation phase followed sequentially with a compensation phase. FIG. 15B illustrates a pulse amplitude, pulse width, dead time, and pulse period of a set of biphasic bipolar pulses.

In some variations, a set of pulses may comprise one or more of a pulse width of between about 240 μs and about 260 μs, a pulse amplitude of up to about 16 mA, a difference between excitation and compensation phase of ±1.5 mA at 16 mA, a dead time of between about 1 μs and about 9 μs, an overshoot of about 8 mA at 16 mA within about 1 μs maximum.

In some variations, an acute treatment session as described herein may include a set of pulses that may comprise a frequency of about 100 Hz, a pulse width of about 250 μs, a pulse duration of about 500 μs, an amplitude increasing at a linear rate of about 0.5 mA/29 sec (e.g., increasing amplitude from about 1.5 mA to about 16 mA in about 14 minutes) followed by a steady amplitude of about 16 mA for about 46 minutes for a total treatment time of about 60 minutes. In some variations, the amplitude at the end of treatment may be linearly decreased from about 16 mA to about 1.5 mA in about 45 seconds and then decreased to 0 mA.

In some variations, a preventative treatment session may include a set of pulses comprising a frequency of about 60 Hz, a pulse width of about 250 μs, a pulse duration of about 500 μs, an amplitude increasing at a linear rate of about 0.5 mA/29 sec (e.g., increasing amplitude from about 0.32 mA or 1.5 mA to about 16 mA in about 14 minutes) followed by a steady amplitude of about 16 mA for about 6 minutes for a total treatment time of about 20 minutes. In some variations, the amplitude at the end of treatment may be linearly decreased from about 16 mA to about 1.5 mA in about 45 seconds and then decreased to 0 mA.

Additionally or alternatively, an input device 120 and/or compute device 160, 162 may be actuated (e.g., by a patient) to manually increase an amplitude (e.g., intensity). For example, each button press may correspond to an increase of about 0.5 mA per second. When a mobile app of a mobile phone is operated, a patient may select to change an amplitude on a GUI. In some variations, a set of pulses (e.g., acute treatment program, preventative treatment program). For example, FIG. is a plot of stimulation intensity (e.g., amplitude mA) over time for a preventative treatment session. The dotted line corresponds to the preventative treatment session as described herein and the solid line at about minute 8 and 10 mA correspond with patient manual control to maintain the intensity. At about minute 13, the patient manually increased the amplitude to about 12 mA until the treatment session ended at about minute 20 with the approximately 45 second ramp down occurring thereafter. It should be appreciated that manual commands may be input by the patient at any point during a treatment session to modify stimulation parameters (e.g., amplitude, duration, etc.).

It should be appreciated that the stimulation system may undergo a startup sequence when power is initiated (e.g., power ON state) In some variations, stimulation system activation may comprise one or more of determining a power level of a power source (e.g., battery), and measuring a predetermined load (e.g., connection of the electrode to a forehead of a patient). In some variations, if a predetermined load is measured, then a predetermined treatment session (e.g., acute treatment session, preventative treatment session) may be automatically initiated unless a command (e.g., a button press, for example, on input device 120) is received to select a different treatment session protocol. The system may transition to a standby state if the predetermined load is not measured.

In some variations, the memory of the system may be configured to store one or more of a session time (e.g., start time, timestamp), a treatment stimulation program selected, a session duration, a maximum current amplitude in a session, a session error, a number of repetitions, a sum of current delivered, a sum of current delivered if maximum current amplitude was reached, a battery charge time, a battery charge duration, a duration to reach full charge, and a battery charge error. In some variations, the data stored in the memory may be encrypted (e.g., AES 128).

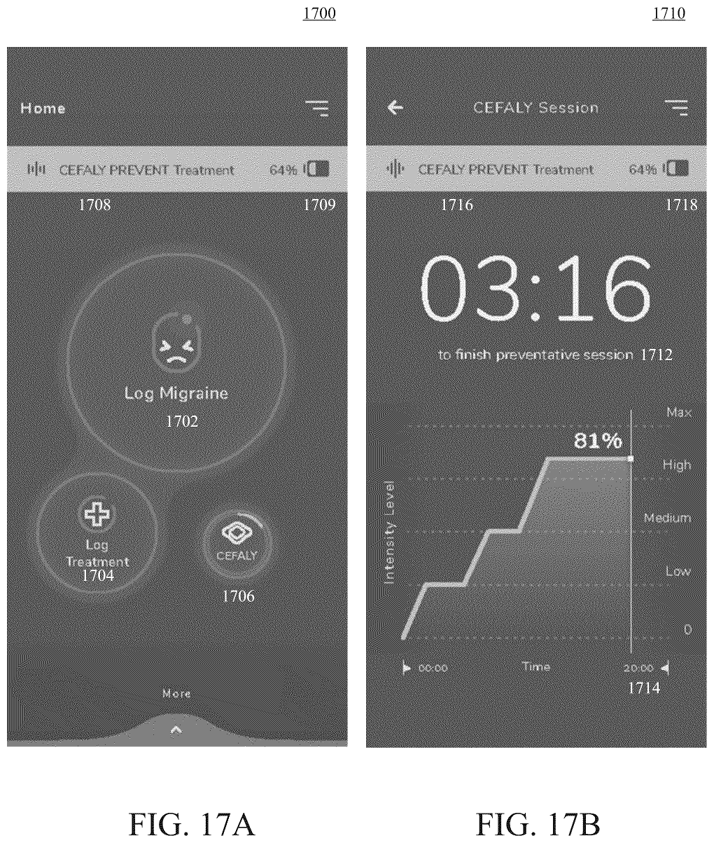

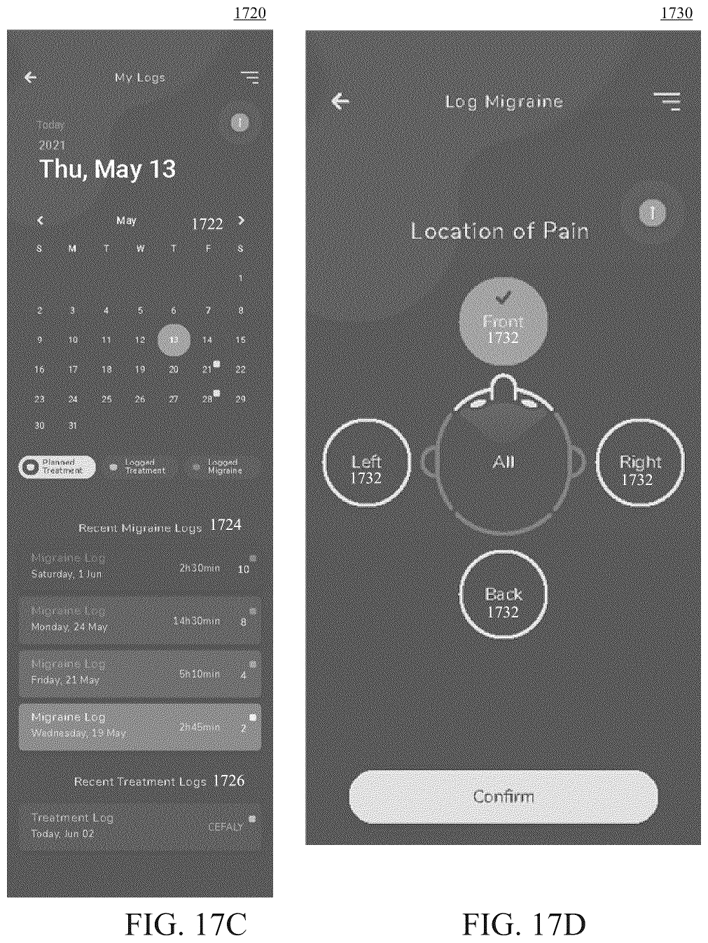

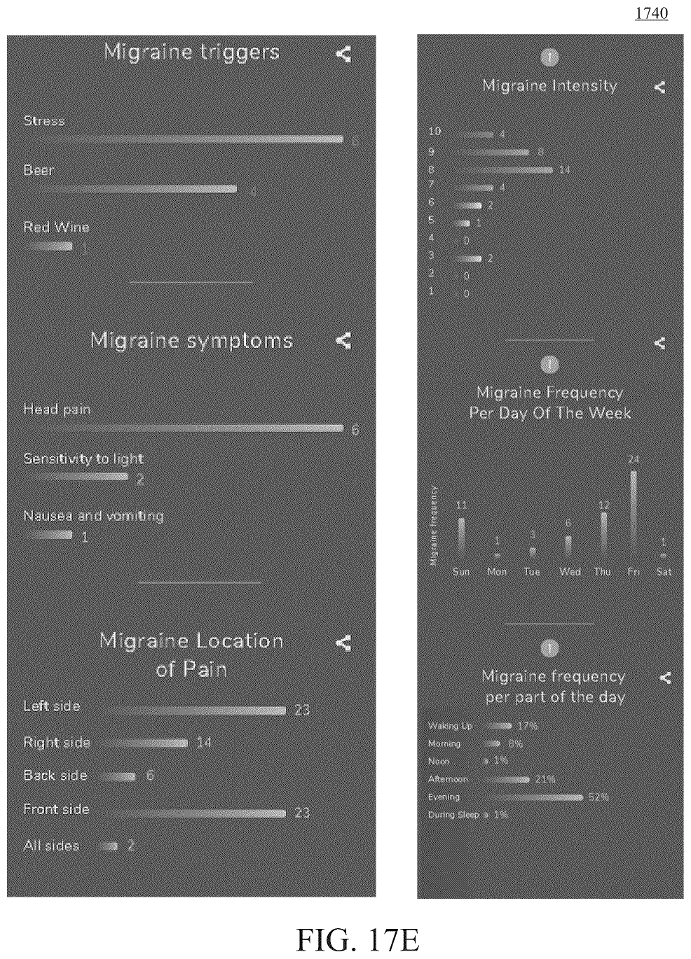

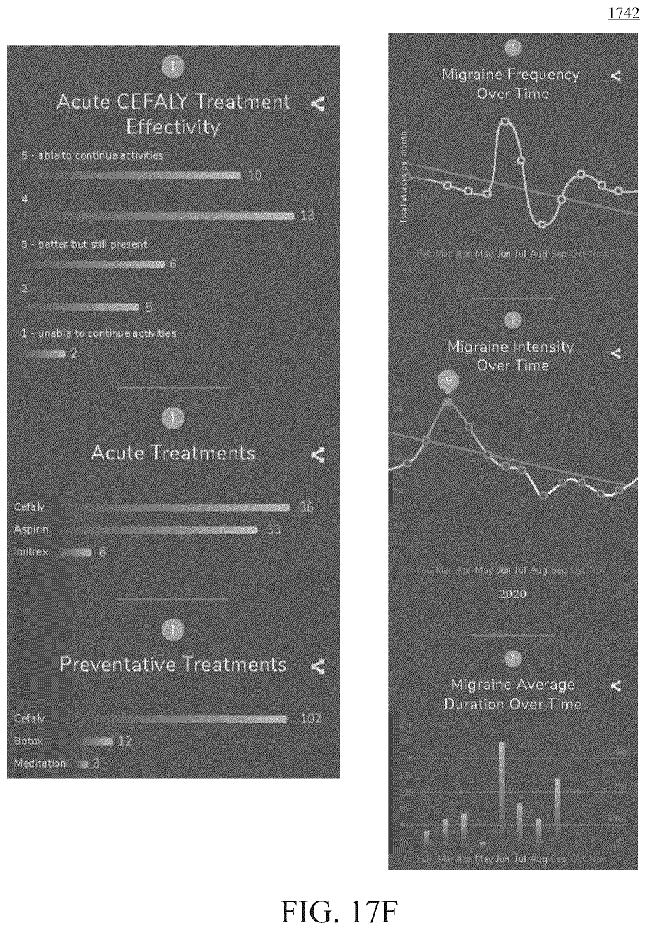

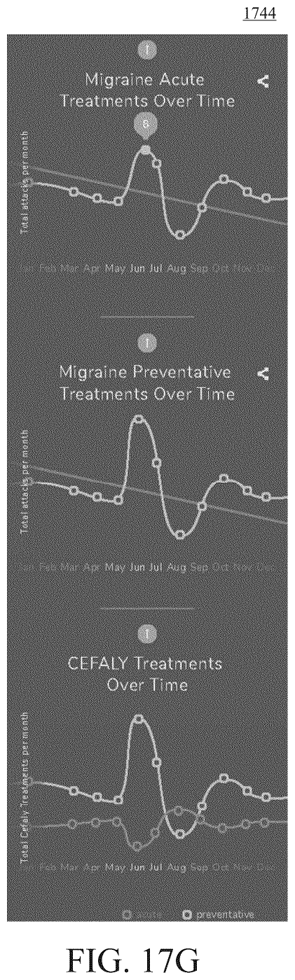

In some variations, a graphical user interface (GUI) may be configured for operating a transcutaneous electrical stimulation system and/or treating a patient. FIGS. 17A-17M depict illustrative variations of a graphical user interface relating to a transcutaneous electrical stimulation interface, as will be described in more detail herein.

B. Electrode Authentication

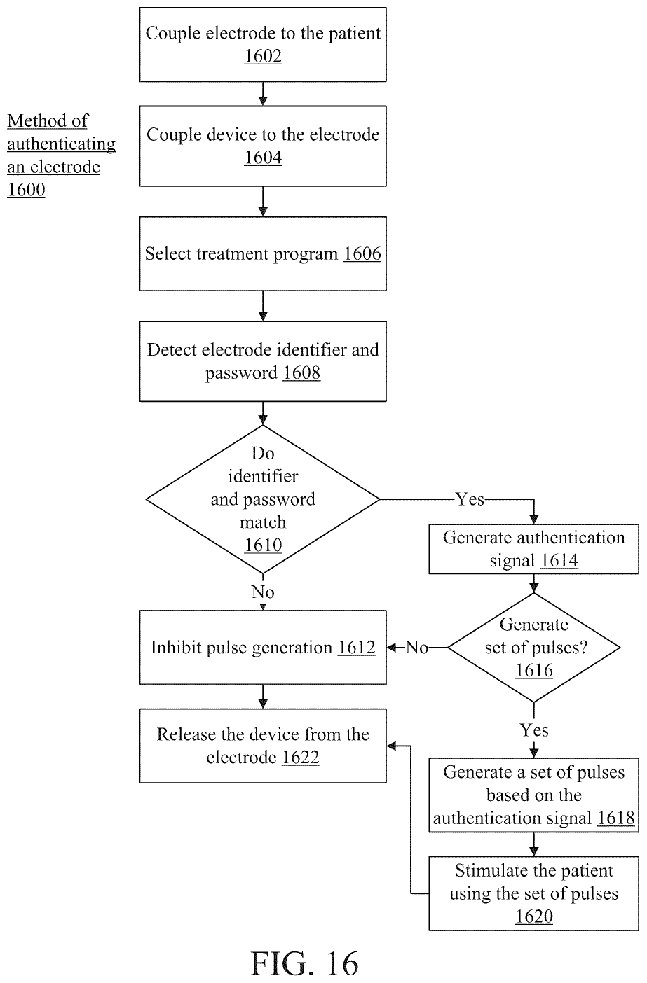

Treatment of a patient using a transcutaneous electrical stimulation system as described herein may include authentication of an electrode prior to generation of electrical stimulation pulses. Generally, the method may include detecting an electrode identifier using the identifier reader, generating an authentication signal based on the detected identifier, and stimulating a trigeminal nerve of a patient using the set of pulses based on the authentication signal. For example, FIG. 16 depicts a flowchart representation that generally describes a method of authenticating an electrode 1600 using any of the systems and devices described herein. The method 1600 may include coupling an electrode to the forehead of a patient. For example, an electrode 112 may comprise an electrode identifier 114 for the electrode 112. A housing of an electrical stimulation system may be coupled to the electrode 1604. For example, the wearable system 100 may comprise a signal generator 116 and an identifier reader 118 (e.g., electrode identifier reader). Optionally, the stimulation system may be connected to a compute device such that the compute device may be configured to control (e.g., operate) the stimulation system. For example, one or more of a Bluetooth and Wi-Fi connection may be established between a mobile application of the compute device and the stimulation system for patient control of the stimulation system using the compute device. In some variations, a treatment program may be selected 1606. For example, the wearable system 100 may be activated with a default treatment program (e.g., acute treatment, preventative treatment). In some variations, the electrode identifier may be detected using the identifier reader of the electrical stimulation system. For example, an electrode identifier (e.g., unique value assigned to the electrode, code) and/or password of the electrode identifier 114 may be detected by the identifier reader 118 of the wearable system 100. The identifier reader 118 may determine if the detected identifier and/or password match an authorized identifier and password 1610. If not, a set of pulses may be inhibited from generation 1612. That is, generation of the set of pulses is inhibited when an authorized electrode identifier is not detected.

If the detected identifier and password match an authorized identifier and password, then an authentication signal is generated based on the detected identifier 1614. A determination of whether to generate the set of pulses may be based on the generated authentication signal 1616. For example, generation of the set of pulses is inhibited 1612 when the authentication signal is one or more of unauthorized, expired, and overused. For example, each electrode may be associated with a predetermined expiration date and a predetermined number of uses (e.g., 5 treatment session, 10 treatment sessions, 20 treatment sessions). Otherwise, a set of pulses is allowed to be generated as a result of the authentication signal indicating a valid electrode 1618. The patient may be stimulated using the set of pulses 1620. For example, a trigeminal nerve of the patient may be stimulated using a set of pulses generated by the signal generator based on the authentication signal indicating that the detected electrode is permitted to be used. In some variations, the system may be released from the electrode 1622. For example, when a treatment session has ended, the stimulation device 102 of the wearable system 100 may be removed from the electrode 112 and/or the electrode 112 may be removed from the skin surface (e.g., forehead) of the patient.

C. Graphical User Interface

In some variations, the stimulation systems described herein may be operated by one or more of an input device on the system itself (e.g., button 310) and a mobile application executed on a compute device(s) 160, 162 (e.g., mobile phone, laptop, desktop PC) in communication with the stimulation system. This may facilitate improved ergonomics, control, and monitoring of a treatment session. In some variations, the mobile application may comprise a set of GUIs as described in more detail herein. FIG. 17A is a variation of a GUI 1700 comprising a menu interface. For example, GUI 1700 may be a home page. The GUI 1700 may comprise one or more of a log disorder icon 1702 (e.g., migraine), a log treatment icon 1704, a stimulation system icon 1706, a treatment status 1708, and a system power indicator 1709 (e.g., system battery status). A user may select one or more of the log disorder icon 1702, log treatment icon 1704, and stimulation system icon 1706 to access additional functionality. For example, selection of the log disorder icon 1702 may transition from the menu interface 1700 to GUI 1730 (e.g., FIG. 17D), selection of log treatment icon 1704 may transition from the menu interface 1700 to GUI 1720 (e.g., FIG. 17C), and selection of the stimulation system icon 1706 may transition from the menu interface to GUI 1710 (e.g., FIG. 17B). In some variations, the stimulation system icon 1706 may include a graphical representation of elapsed treatment time. For example, in some variations, the stimulation system icon 1706 may indicate an amount of elapsed treatment time as a ring that fills up (e.g., extends along its circumference) over time, as a series of icons (e.g., bars) that progressively appear, as a line that increases in length, a combination thereof, or the like. In some variations, different colors may be used to indicate different portions of the treatment session. For example, yellow, green, and red may be used to indicate respective ramp-up, steady state, and ramp-down current intensity states of a treatment session.

FIG. 17B is a variation of a GUI 1710 comprising a treatment interface. For example, GUI 1700 may display data corresponding to an in-progress stimulation treatment session. The GUI 1710 may comprise one or more of a timer 1712 (e.g., indicating an amount of time elapsed and/or remaining time in a treatment session), a plot 1714 corresponding to intensity (e.g., current delivered) over time, treatment status 1716 (e.g., acute treatment session, preventative treatment session), and system power indicator 1718 (e.g., percentage of battery life remaining).

FIG. 17C is a variation of a GUI 1720 comprising a log treatment interface. For example, GUI 1720 may include a calendar 1722 containing information related to one or more planned treatment sessions, logged treatment sessions, and/or logged disorders. The GUI 1720 may comprise one or more of a timer 1712 (e.g., indicating an amount of time elapsed and/or remaining time in a treatment session), a plot 1714 corresponding to intensity (e.g., current delivered) over time, a treatment status 1716, and a system power indicator 1718. The GUI 1720 may further comprise one or more of a disorder summary 1724 (e.g., migraine) and a treatment summary 1725. A disorder summary 1724 may include a set of logged disorder data including, but not limited to, date, duration, intensity, and patient input description. A treatment summary 1726 may comprise electrical stimulation data comprising one or more of a session time, a treatment stimulation program selected, a session duration, a maximum current amplitude in a session, a session error, a number of repetitions (e.g., number of treatments), a sum of current delivered, and a sum of current delivered if maximum current amplitude was reached. Additionally or alternatively, a system summary may be displayed and comprise one or more of a battery charge time, a battery charge duration, a duration to reach full charge, and a battery charge error. Treatment sessions initiated by the electrical stimulation systems described herein may be automatically logged into the log treatment interface and viewed at a later time.

FIG. 17D is a variation of a GUI 1730 comprising a log disorder interface. For example, GUI 1730 may display a set of selectable location icons 1732 corresponding to pain locations on a patient's head. Additionally or alternatively, a patient may input one or more of a perceived intensity of a disorder (e.g., from 1-10), a trigger (e.g., food, stress, smell), one or more symptoms (e.g., pulsating head pain, body pain, distortion in vision), one or more medications taken (e.g., prescribed medication, over-the-counter medications such as NSAIDs), a non-drug treatment (e.g., electrical stimulation session), an effectiveness of any of the foregoing medications and/or treatments (e.g., from 1-5, able/unable to continue activity). In some variations, the patient may further input a description of the disorder as one or more of text (e.g., journal), audio (e.g., voice recording), and/or image (e.g., picture, video, selfie). The data input to the log disorder interface may be tracked over time for the patient to monitor.

In some variations, one or more notification may be displayed on a compute device 160 of a user 140 of the system. For example, a predetermined reminder for preventative treatment may be output on one or more of a compute device 160 and wearable system 100 at predetermined intervals (e.g., every day at 9:00 a.m. for a treatment time of 20 minutes).

FIGS. 17E-17G are variations of GUIs 1740, 1742, 1744 comprising disorder and treatment trends. For example, GUI 1740 may display a set of disorder trends such as patient input triggers, symptoms, locations, intensities, as well as calculated trends such as disorder and/or treatment frequency (e.g., per day of the week, per part of the day). GUI 1742 may display one or more of treatment effectiveness, treatment programs (e.g., acute, preventative), and disorder trends over time (e.g., migraine frequency, migraine intensity, migraine average duration). GUI 1744 may display one or more treatment trends over time (e.g., acute treatment, preventative treatment, electrical stimulation system treatment). It should be appreciated that the data displayed in any of the GUIs may be transmitted (e.g., exported) as a separate report for one or more of a patient, family member, caregiver, health care professional, and the like. In some variations, the data displayed in any of the GUIs disclosed herein may be displayed in real-time to an authorized user (e.g., patient, family member, caregiver, health care professional). For example, a health care professional may be granted access to view GUIs and/or reports corresponding to treatment trends, disorder trends, and the like.

While described above in relation to the treatment of migraines, it should be appreciated that GUIS 1700-1744 with corresponding features may be utilized in relation to the treatment of any of the disorders or conditions described herein. For example, a GUI may comprise a sleep log icon where the patient may input their own sleep data (e.g., description of sleep, restfulness, activities before sleep) or an anxiety log where a patient may input anxiety triggers, anxiety intensity, anxiety frequency, duration, anxiety symptoms, and the like. Similarly a GUI may comprise one or more icons related to patient data including one or more of heart rate, oxygen saturation, blood pressure, menstrual cycle, sleep, nutrition, hydration, medications consumed, activity level, geolocation, weather, air pressure, temperature, humidity, pollen count, air quality, pollution levels, demographic data, and the like. Patient data may be received from a measurement device configured to measure, receive, and/or analyze one or more characteristics of a user. Non-limiting examples of measurement devices include a wearable activity device (e.g., pedometer or other activity tracker, smart jewelry, smart watch, smart ring), a hydration tracker, a blood pressure monitor, a heart rate monitor, an ultrasonic sensor, a cholesterol monitor, a scale, geolocation devices (e.g., GPS, GLONASS), a smartphone, a refrigerator, a PC, an implantable diagnostic device, an ingestible diagnostic device, and other diagnostic devices. Furthermore, while in some variations the measurement device may be a wearable device as described above, it should be understood that in other variations, the measurement device may be configured as a non-wearable device. One example of a non-wearable measurement device for measuring one or more sleep parameters is an implantable device or an external monitor such as a bedside monitor or home virtual assistant device (e.g., similar to Amazon Echo® or Google Home™ devices), a set top box service (e.g., similar to Apple TV®), or other smart appliances such as a clock, radio, and the like. In some variations, systems described herein may comprise a plurality of measurement devices, one or more of which may be a wearable device and one or more of which may be configured as a non-wearable device.

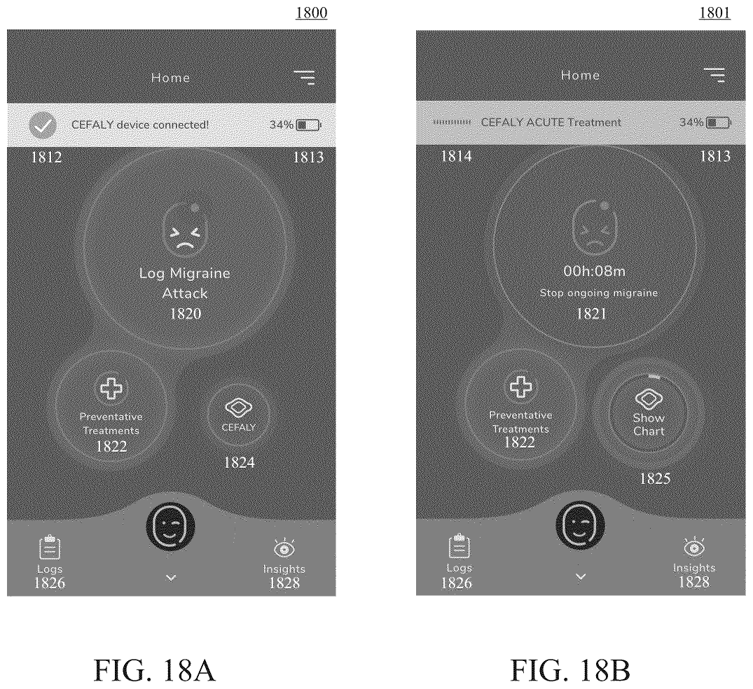

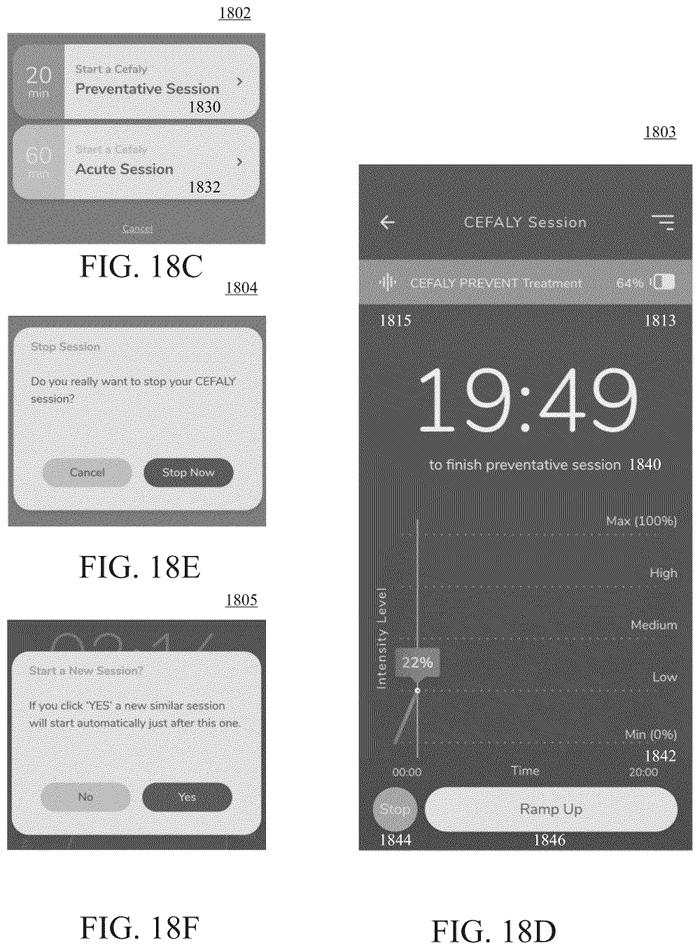

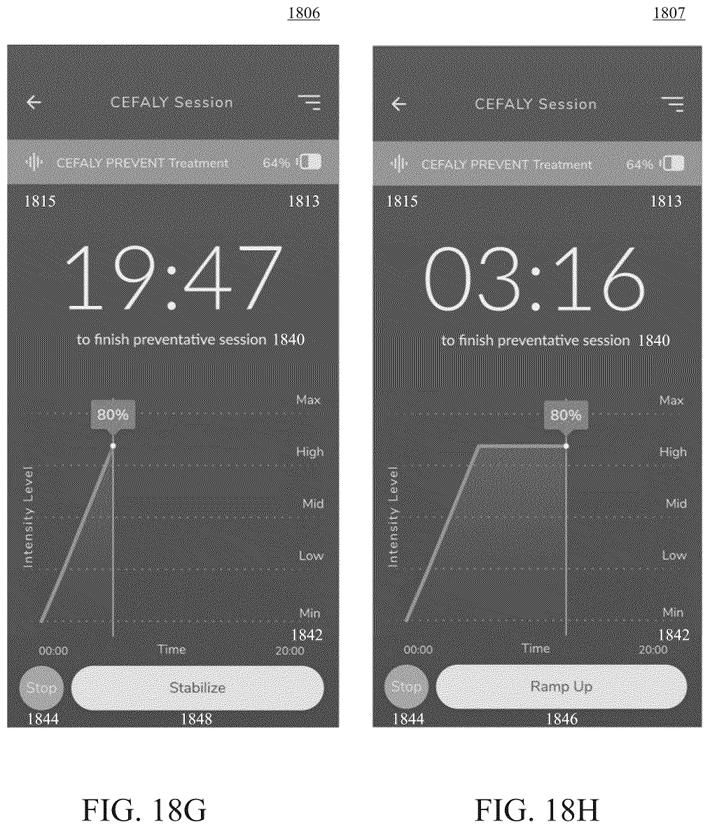

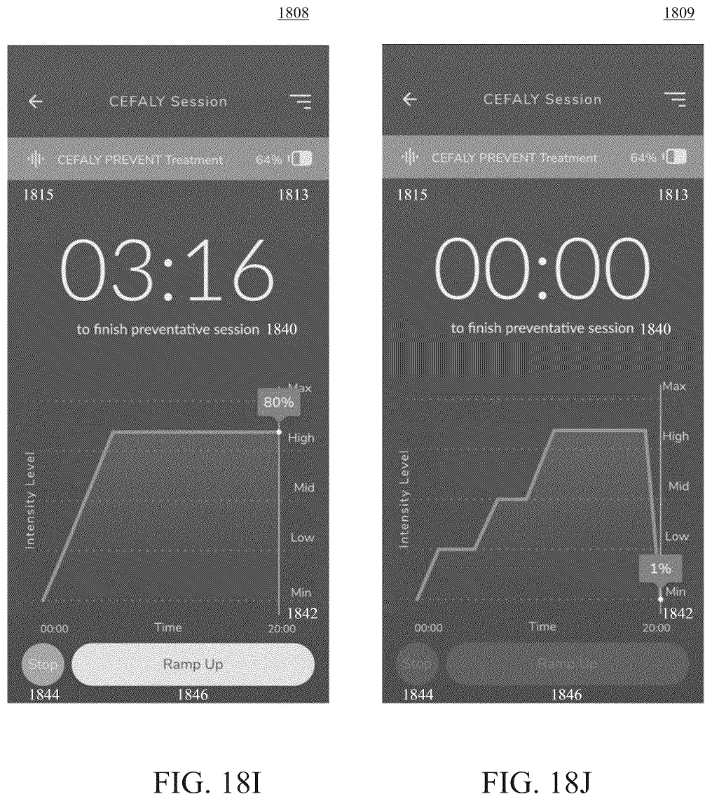

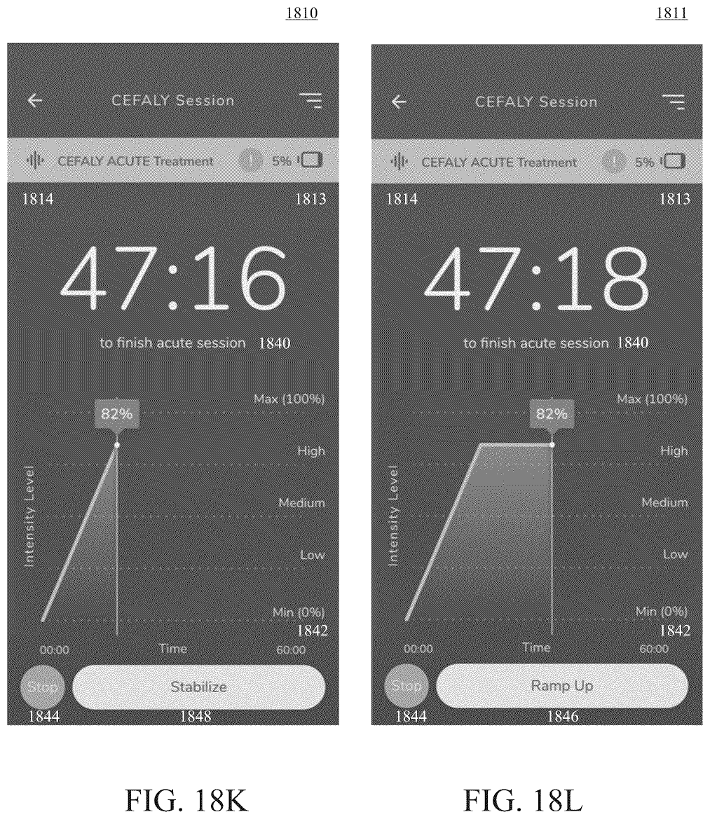

FIG. 18A is a variation of a GUI 1800 comprising a menu interface. For example, GUI 1800 may be a home page. The GUI 1800 may comprise one or more of an acute treatment icon 1820, a preventative treatment icon 1822, a stimulation system icon 1824, a log disorder icon 1826, an insight icon 1828, a stimulation system connection status 1812, and a system power indicator 1813 (e.g., system battery status). A user may select one or more of the acute treatment icon 1820, preventative treatment icon 1822, and stimulation system icon 1824 to access additional functionality. For example, selection of the acute treatment icon 1820 may initiate and/or begin a timer to track, log, and/or control an acute treatment using a stimulation device (e.g., device 102, 200, 202, 204, 300, 400) and transition from the GUI 1800 to the GUI 1801 (e.g., FIG. 18B). Selection of the preventative treatment icon 1822 may initiate and/or begin a timer to track, log, and/or control a preventative treatment using the stimulation device. Selection of the stimulation system icon 1824 may transition from the GUI 1800 to the GUI 1802 (e.g., FIG. 18C). The GUI 1802 may include a preventative treatment icon 1830 for initiating a preventative treatment using the stimulation device and an acute treatment icon 1832 for initiating an acute treatment using the stimulation device. Any of the icons described herein may include one or more of (including a combination of) alphanumeric characters, pictures, graphical elements or the like. Additionally or alternatively, the icons may be the same size or may have variable sizes, and the sizes and prominence of the icons on the GUI may change depending on a user's selection. For example, in some variations, the selected icon (in FIG. 18A, the acute treatment icon 1820) may be larger than the other icons.

Selection of the log disorder icon 1826 in GUIs 1800, 1801 may transition from the GUIs 1800, 1801 to the GUI 1720 (e.g., FIG. 17C). Selection of the insight icon 1828 may transition from the GUIs 1800, 1801 to one or more of the GUIs 1740, 1742, 1744 (e.g., FIG. 17E-17G).

In some variations, selection of the acute treatment icon 1820 may activate and/or begin a timer to track and/or log an acute treatment using a stimulation device and transition the GUI 1800 to the GUI 1801. The GUI 1801 may include an acute treatment icon 1821 including a timer and a treatment status 1814 indicating that an acute treatment using the stimulation device is active. Additionally or alternatively, a stimulation system icon 1825 may include a graphical representation (e.g., ring-based timer) of elapsed treatment time or remaining treatment time. Moreover, while depicted in relation to the stimulation system icon 1825, it should be appreciated that the acute treatment icon 1821 and/or the preventative treatment icon 1822 (when activating and/or timing a preventative treatment) may include a graphical representation (e.g., ring-based timer) of elapsed treatment time or remaining treatment time.

FIGS. 18D and 18G-18J are variations of a graphical user interface comprising a preventative treatment interface. FIG. 18D is a variation of a GUI 1803 comprising a preventative treatment interface. For example, GUI 1803 may display data corresponding to an in-progress (e.g., real-time) preventative stimulation treatment session. The GUI 1803 may comprise one or more of a timer 1840 (e.g., indicating an amount of time elapsed and/or remaining time in a treatment session), a plot 1842 corresponding to intensity (e.g., current delivered) over time, stop treatment icon 1844, increase intensity icon 1846, treatment status 1815 (e.g., preventative treatment session), and system power indicator 1813 (e.g., percentage of battery life remaining). An intensity level of the stimulation treatment may correspond to a pulse amplitude of a treatment stimulation program generated by a stimulation device and delivered to the patient. For example, a treatment stimulation program (e.g., preventative treatment program) may comprise a predetermined minimum pulse amplitude (e.g., 0 mA) and a predetermined maximum pulse amplitude (e.g., about 16 mA). The plot 1842 may depict the delivered pulse amplitude between the minimum pulse amplitude (e.g., 0 mA) and the maximum pulse amplitude on a predetermined scale (e.g., min—0%, low—25%, medium—50%, high—75%, max—100%) over time (e.g., minutes). For some patients, presenting intensity level as a function of current may be unfamiliar and confusing such that a relative description of the treatment intensity (e.g., low, medium, high) may be more understandable and useful.

In some variations, a patient may select the stop treatment icon 1844 to inhibit further stimulation (e.g., stop the treatment session) during a treatment session and/or stop tracking and/or logging a treatment session. For example, selection of the stop treatment icon 1844 may inhibit preventative treatment or acute treatment using a stimulation device (e.g., device 102, 200, 202, 204, 300, 400) and/or may stop tracking and/or logging a preventative or acute treatment, and may transition the displayed GUI to the GUI 1804 (e.g., FIG. 18E). The patient may confirm the end of the treatment session via GUI 1804. In some variations, the GUI 1804 may be displayed over another GUI (e.g., preventative treatment interface, acute treatment interface).

In some variations, an objective of preventative stimulation treatment may be to gradually acclimate the patient to higher intensity treatment over time (e.g., over a plurality of treatment sessions) in order to prevent or reduce the likelihood of a migraine. Acclimation is generally a patient-specific process. For example, a patient may initially find a preventative treatment intensity above a low level (e.g., 25% of a maximum intensity level) for more than a few minutes to be uncomfortable or intolerable. However, the patient may become acclimated to the low intensity level over multiple treatment sessions such that the patient may be able to tolerate a higher level of intensity after an acclimation period.

In some variations, a patient may modify the predetermined set of pulses delivered during a stimulation treatment session to improve their treatment outcomes (e.g., reduce the frequency and intensity of migraines). For example, a patient may modify the predetermined set of pulses including modifying one or more of: the pulse amplitude, ramp up time, steady time, ramp down time, and session duration. In some variations, a preventative stimulation treatment session may comprise a predetermined set of pulses configured to prevent a migraine. As a non-limiting example of preventative stimulation parameters, a predetermined set of pulses may comprise a pulse frequency of about 60 Hz, a pulse width of about 250 μs, a pulse period of about 500 μs, a maximum pulse amplitude of about 16 mA, a ramp up time of about 14 minutes, a steady time of about 6 minutes, a ramp down time of about 45 seconds, and a session duration of about 20 minutes. In GUI 1803, a patient may select the increase intensity icon 1846 to increase a rate that the intensity (e.g., pulse amplitude) increases. Increasing the intensity rate may reduce the ramp up time of a treatment session and enable a longer steady time without modifying the session duration. Additionally or alternatively, the patient may select a decrease intensity icon (not shown) to decrease a rate at which the intensity increases. This may be useful when the patient desires a longer ramp up time where the intensity increases more slowly (i.e., at a lower rate).

FIG. 18G is a variation of a GUI 1806 comprising a preventative treatment interface where a delivered pulse amplitude has linearly increased from 0% to about 80% of a maximum pulse amplitude. In GUI 1806, a patient may select the maintain intensity icon 1848 to maintain a current pulse amplitude (e.g., 80% of a maximum pulse amplitude). This may be useful when the patient determines that a further increase in intensity (e.g., pulse amplitude) is undesirable or would be poorly tolerated (e.g., uncomfortable, painful). FIGS. 18H and 181 are variations of respective GUIs 1807, 1808 comprising a preventative treatment interface where the intensity has been maintained (e.g., at 80% of a maximum pulse amplitude) for a steady time. In some variations, a patient may select the increase intensity icon 1846 during the steady time to increase the maximum pulse amplitude of the preventative treatment session. Additionally or alternatively, the patient may select a decrease intensity icon (not shown) if a lower pulse amplitude is desired. FIG. 18J is a variation of a GUI 1809 comprising a preventative treatment interface for a completed preventative treatment session. The plot 1842 of the GUI 1809 depicts a staircase-like increase in intensity over time with alternating ramp up periods (increasing intensity) and steady state periods (constant intensity), and a ramp down period (decreasing intensity). In some variations, one or more of an audio notification and haptic notification may be output during a treatment session (e.g., upon a change in intensity, upon start and/or stop of a ramp up period, upon start and/or stop of a ramp down period, upon start and/or stop of a steady state period) and/or upon completion of a treatment session. In some variations, selection of the stop treatment icon 1844 and the increase intensity icon 1846 may be inhibited upon completion of a treatment session.

In some variations, completion of a treatment session may transition the displayed GUI (e.g., GUI 1809) to the GUI 1805 (e.g., FIG. 18F). The patient may confirm to begin another treatment session via the GUI 1805. In some variations, the GUI 1805 may be displayed over (overlaid) another GUI (e.g., GUI 1809).

FIGS. 18K and 18L are variations of a graphical user interface comprising an acute treatment interface. In some variations, an objective of acute stimulation treatment is to shorten one or more of a duration and intensity of an acute (e.g., in-progress) migraine. FIG. 18K is a variation of a GUI 1810 comprising an acute treatment interface. For example, GUI 1810 may display data corresponding to an in-progress (e.g., real-time) acute stimulation treatment session. The GUI 1810 may comprise one or more of a timer 1840 (e.g., indicating an amount of time elapsed and/or remaining time in a treatment session), a plot 1842 corresponding to intensity (e.g., current delivered) over time, stop treatment icon 1844, maintain intensity icon 1848, treatment status 1814 (e.g., acute treatment session), and system power indicator 1813 (e.g., percentage of battery life remaining). In some variations, a patient may select the stop treatment icon 1844 to inhibit further stimulation (e.g., stop the treatment session) during a treatment session.

In some variations, a patient may modify the predetermined set of pulses delivered during an acute stimulation treatment session to improve their treatment outcomes (e.g., reduce a duration of a migraine). GUI 1810 depicts a plot 1842 where a delivered pulse amplitude has linearly increased from 0% to about 82% of a maximum pulse amplitude. In GUI 1803, a patient may select the maintain intensity icon 1848 to maintain a current pulse amplitude (e.g., 82% of a maximum pulse amplitude) where, for example, the 82% of maximum pulse amplitude is sufficient to mitigate migraine symptoms.

FIG. 18L is a variation of a GUI 1811 comprising a preventative treatment interface where the intensity has been maintained (e.g., at 82% of a maximum pulse amplitude) for a steady time. In some variations, a patient may select the increase intensity icon 1846 during the steady time to increase the maximum pulse amplitude of the acute treatment session if additional stimulation is desired by the patient. Additionally or alternatively, the patient may select a decrease intensity icon (not shown) if a lower pulse amplitude is desired.

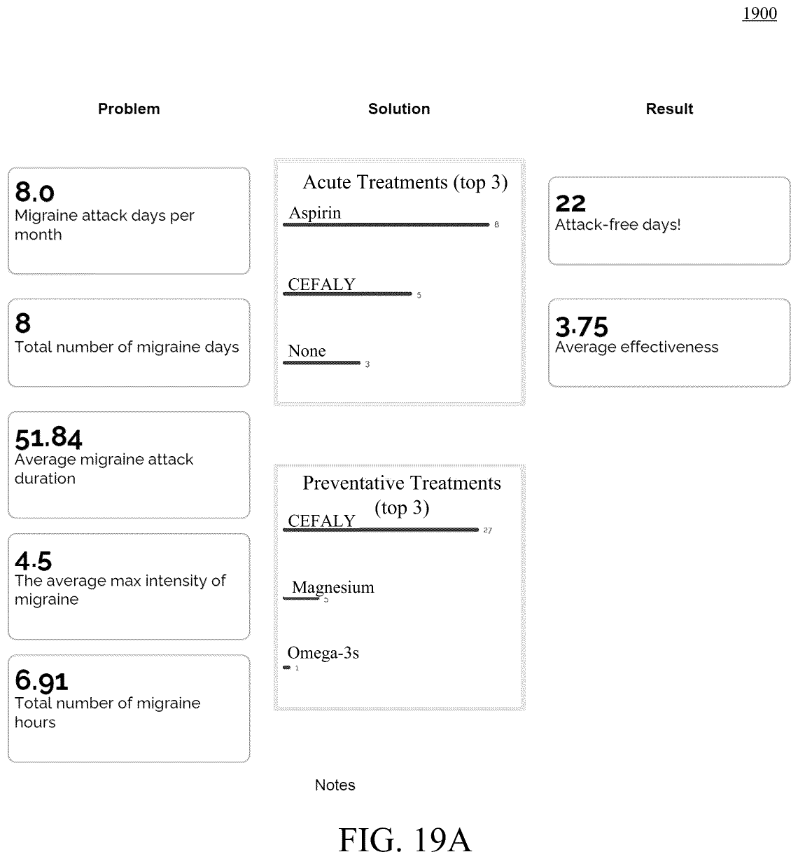

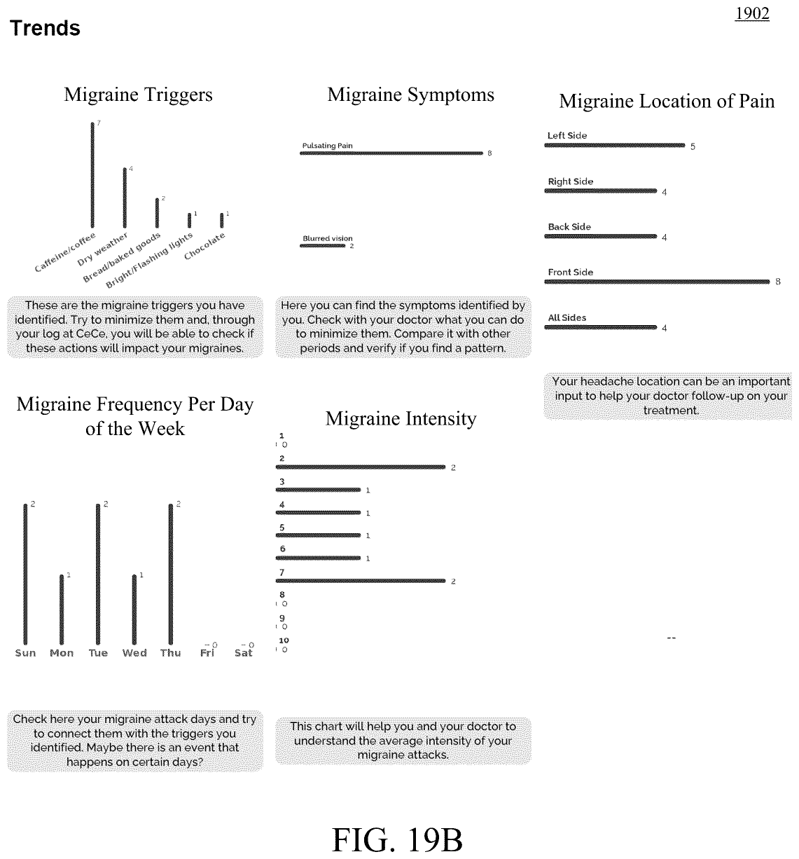

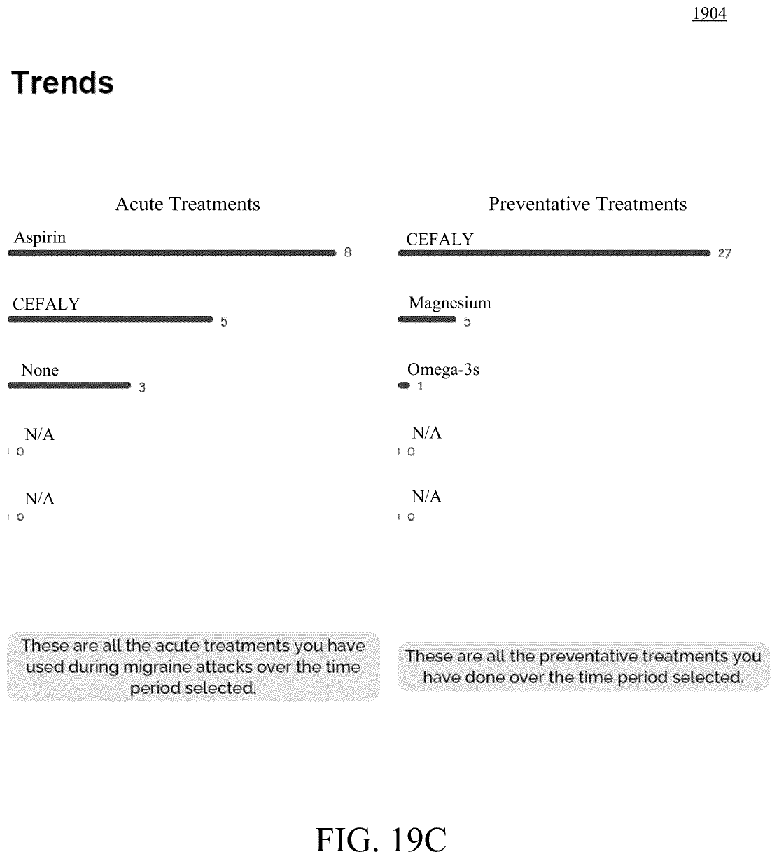

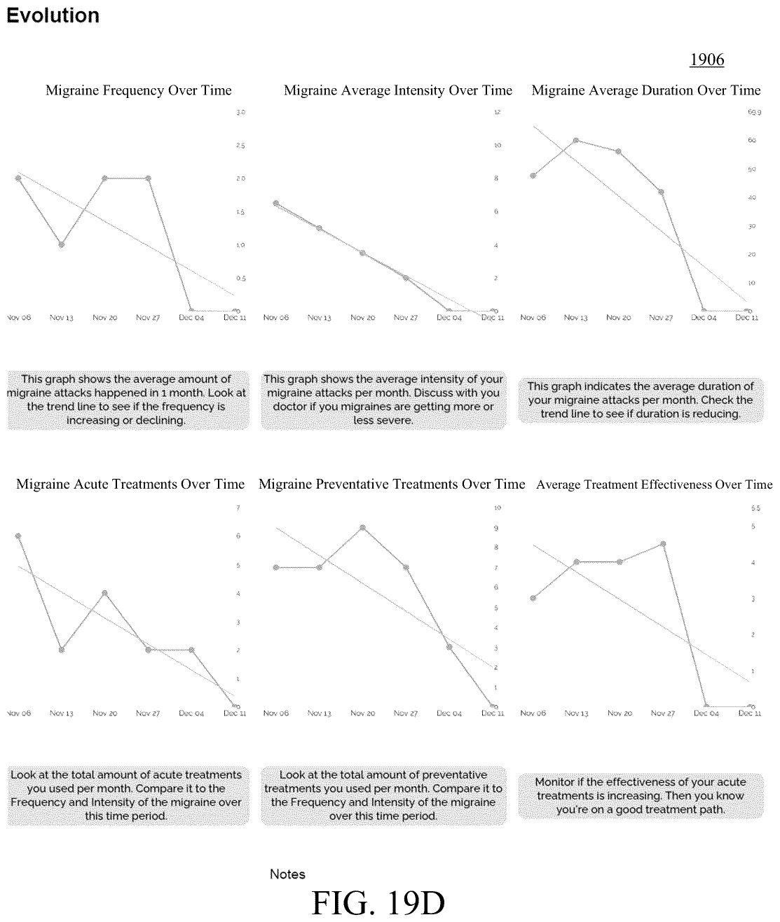

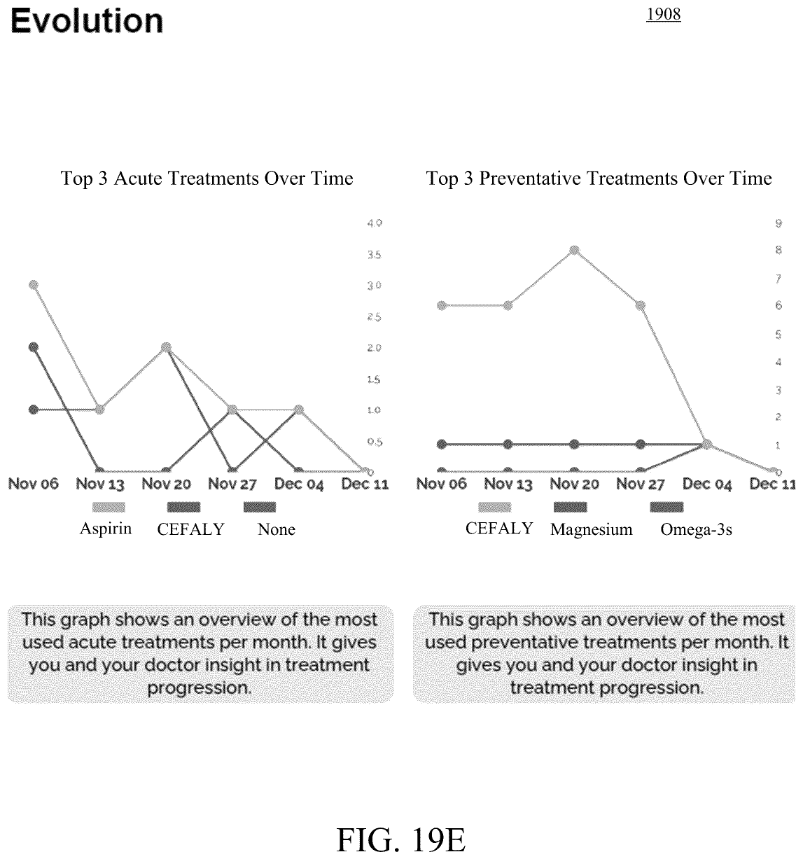

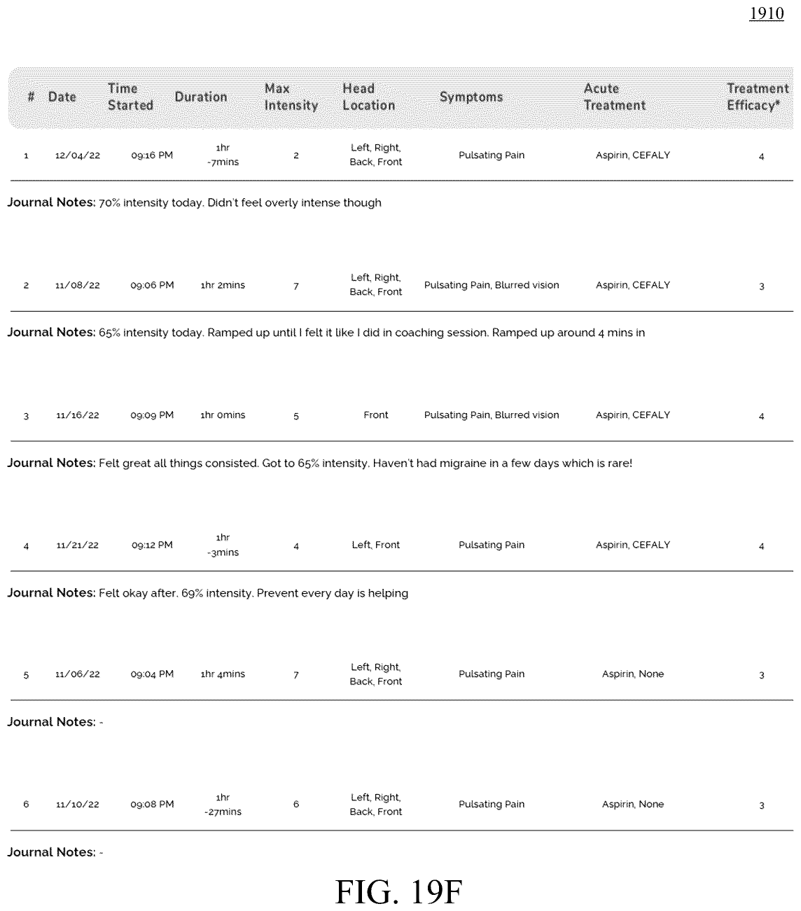

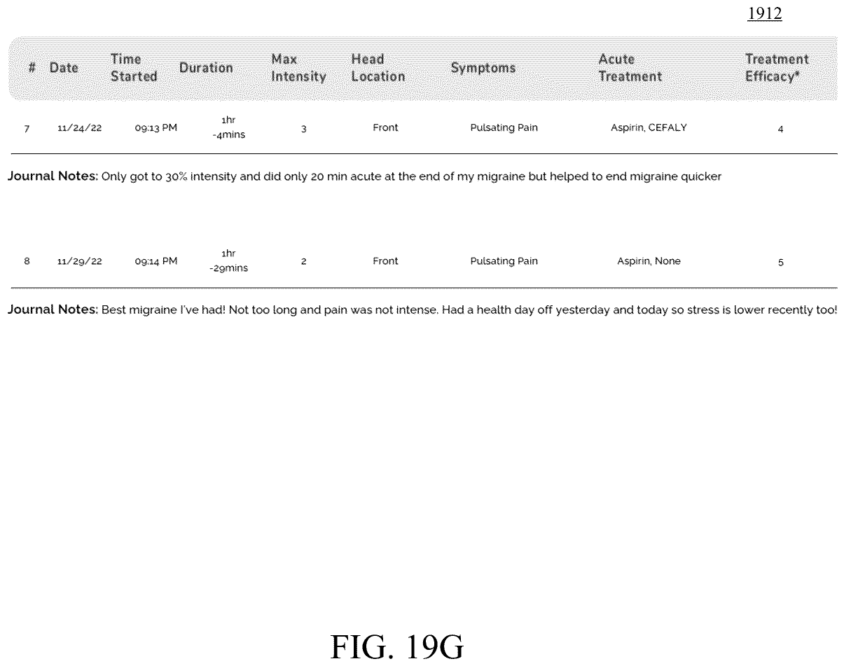

FIGS. 19A-19G depict illustrative variations of a graphical user interface 1900, 1902, 1904, 1906 comprising disorder and treatment trends. For example, GUI 1900 may include a “Problem” column including disorder trends (e.g., “8.0 migraine attack days per month”), a “Solution” column including treatment statistics, and a “Result” column including treatment trends (e.g., “22 Attack-free days!”). The GUI 1902 may display one or more of disorder trends over time (e.g., migraine triggers, migraine symptoms, migraine location, migraine frequency, migraine intensity). The GUI 1904 may display one or more treatment trends over time (e.g., acute treatment, preventative treatment, electrical stimulation system treatment, medication, no treatment). The GUIs 1906, 1908 may display a set of disorder and treatment trends over time (e.g., migraine frequency, migraine intensity, migraine duration, treatment frequency, treatment effectiveness). The GUIs 1900, 1902, 1904, 1906 may be accessible by one or more of a patient and authorized user (e.g., family member, caregiver, health care professional). The GUIs 1910, 1912 may display a set of disorder and treatment data including number, date, time started, duration, maximum intensity, location, symptom, acute treatment, efficacy, and user notes.

D. Dosage Determination

Treatment of a patient using a transcutaneous electrical stimulation system, for example, one as described herein, may include determining dosage and modifying a treatment program (e.g., stimulation parameters) based on the determined dosage. Determining neurostimulation dosage over time for modifying stimulation parameters may facilitate optimizing treatment for a disorder, by, for example, facilitating providing a lowest effective dose of neurostimulation to the patient, thereby improving treatment outcomes and minimizing the risk of adverse effects of the treatment. Neurostimulation comprises applying electrical stimulation having a plurality of stimulation parameters including, but not limited to, frequency, current (e.g., pulse amplitude), pulse width, and the like. However, these parameters do not provide a dosage like conventional physical drug dosages (e.g., take 20 mg twice daily, 10 mL of oral medication with every meal). Therefore, determining a neurostimulation dosage for a patient using an intuitive metric may facilitate understanding and modification of neurostimulation treatment, and may assist in optimizing neurostimulation treatment for a particular patient, including, for example, reducing total neurostimulation dosage over time.

For example, Table 1 depicts a conventional summary of neurostimulation treatment for an exemplary patient:

TABLE 1

| | Preventative | Acute |
|---|---|---|
| | treatment program | treatment program |
| Number of treatments/month | 15 | 5 |
| Average treatment intensity | 10 mA | 6 mA |
| Average treatment duration | 10:54 | 54:12 |
| (minutes) | | |

Based on the data in Table 1, a patient may be unable to determine if the dosage they are receiving from the preventative and acute treatment programs are appropriate or how they can be modified for improvement. For example, even determining which treatment program provides a higher dosage between the preventative treatment program and the acute treatment program may not be apparent for patients and/or health care providers from Table 1 alone. While the acute treatment program provides a lower number of treatments and a lower average treatment intensity relative to the preventative treatment program, the average treatment duration of the acute treatment program is five times higher than the preventative treatment program. The average treatment duration corresponds to a session duration but generally include time where energy is not delivered (e.g., dead time between pulses). The methods described herein provide a baseline measure (e.g., metric) for neurostimulation dosage useful for determining and modifying neurostimulation treatment.

In some variations, a dosage of electrical stimulation of a neurostimulation treatment may be determined by calculating an electric charge delivered to an anatomical target, such as, for example a nerve (e.g., the trigeminal nerve) of the patient. For example, the electric charge may be calculated based on the stimulation parameters of the electrical stimulation, including one or more of a frequency, a current, a pulse width, a pulse amplitude (e.g., stimulation intensity), a dead time (e.g., time without energy delivery), a pulse duration, and a session duration. A dosage may be calculated based on the energy delivered to the patient over time (e.g., amplitude and stimulation duration excluding periods between pulses where no energy is delivered). For example, a total duration of a preventative treatment program may be about 20 minutes, but a stimulation duration of the preventative treatment program where energy is actually delivered (e.g., excluding the time periods of a session duration where energy is not delivered such as between pulse widths) may be about 50 seconds within the 20 minute treatment program. Similarly, a total duration of an acute treatment program may be about 60 minutes, but a stimulation duration of the acute treatment program may be about 90 seconds within the 60 minute treatment program. The amplitude of stimulation may vary (e.g., ramp up, stabilize, ramp down) within the stimulation duration, as shown for example in GUI 1809 of FIG. 18J.

In some variations, a dosage of electrical stimulation may be calculated in terms of electric charge (e.g., coulomb C, centicoulomb cC) as a sum of current amplitude multiplied by a corresponding time duration that the current is being delivered. In some variations, a preventative treatment program may comprise a dose of up to about 40 cC per treatment session, between about cC and about 25 cC per treatment session, between about 10 cC and about 35 cC per treatment session, and between about 15 cC and about 25 cC per treatment session, including all values and sub-ranges in-between.

In some variations, an acute treatment program may comprise a dose of up to about 200 cC per treatment session, between about 10 cC and about 150 cC per treatment session, between about cC and about 140 cC per treatment session, between about 30 cC and about 100 cC per treatment session, and between about 100 cC and about 200 cC per treatment session, including all values and sub-ranges in-between.

Table 2 depicts a summary of neurostimulation treatment over time including corresponding neurostimulation dosages:

TABLE 2

| | Preventative | | Acute | |
|---|---|---|---|---|
| | treatment program | | treatment program | |
| Number of treatments/month | 15 | | 25 | |
| Average treatment intensity | 10 | mA | 6 | mA |
| Average treatment duration | 10:54 | | 54:12 | |
| (minutes) | | | | |
| Average dose/treatment | 4.9 | cC | 46 | cC |
| Average dose/month | 73.9 | cC | 1,150 | cC |

Table 2 includes the same data as Table 1 but additionally dose data including an average dose per treatment and average dose per month calculated based on the stimulation parameters of the electrical stimulation provided during the respective preventative and acute treatment programs. For example, based on Table 2, an average dose per treatment of the acute treatment program (e.g., 46 cC) is more than nine times the average dose per treatment of the preventative treatment program (e.g., 4.9 cC). The ratio of average dose per month between the acute treatment program and preventative treatment program is even greater at about 15.6 (e.g., 1,150 cC/73.9 cC). This disparity in total dose delivered between the acute treatment program and the preventative treatment program may be largely attributed to the higher number of acute treatments performed relative to preventative treatments.

Based on the additional context and insight these determined dosages provide, one or more modifications may be made to a patient's neurostimulation treatment program to improve treatment outcomes. For example, based on the calculated dosages in Table 2, the patient and/or health care provider may determine that the total dose being applied via the acute treatment program (e.g., 1,150 cC per month) presents an undesirable risk level for adverse effects and may modify the neurostimulation treatment program (e.g., one or more stimulation parameters) to increase one or more of the session frequency, treatment duration, treatment intensity, and number of preventative treatment sessions. In some variations, one or more of a preventative treatment program session frequency and dosage may be increased based on the determined dosage and an acute treatment program session frequency may be reduced as described in detail with respect to Table 3. In this manner, a total dosage applied to the patient may be reduced over a subsequent predetermined period of time. For example, after performing the modified treatment, a summary of neurostimulation is reflected in Table 3.

TABLE 3

| | Preventative | | Acute | |
|---|---|---|---|---|
| | treatment program | | treatment program | |
| Number of treatments/month | 28 | | 7 | |
| Average treatment intensity | 10 | mA | 6 | mA |
| Average treatment duration | 19:54 | | 45:12 | |
| (minutes) | | | | |
| Average dose/treatment | 13 | cC | 38 | cC |
| Average dose/month | 364 | cC | 266 | cC |

Between Table 2 and Table 3, the number of preventative treatment sessions increased from 15 to 28 while the number of acute treatment sessions decreased from 25 to 7, as the need for acute migraine treatment naturally decreased due to the effectiveness of the modified treatment program. That is, as the patient engages in more preventative treatment sessions with a longer treatment duration, the need for acute treatment sessions may naturally decrease in frequency. Although the average dose per treatment and average dose per month of the preventative treatment program increased from Table 2 to Table 3, the average dose per treatment and average dose per month of the acute treatment program decreased such that the total dose per month delivered between the treatment programs in Table 2 and Table 3 reduced by almost half from 1223.9 cC to 630 cC. Accordingly, determining a dosage of electrical stimulation applied to the anatomical target, in this example, the trigeminal nerve, may facilitate stimulation parameter modification for achieving a lower/lowest effective dose and decreased occurrence of disorder symptoms, in this example, migraines. The dosage determination described herein may be used for any anatomical target and any disorder that utilizes electrical stimulation for treatment.

In some variations, modification of stimulation parameters to improve treatment outcomes may further include generating additional treatment programs based on, for example, patient and/or health care professional input. For example, a patient may determine from Table 2 that a frequency and a duration of a preventative treatment program session should be increased in order to reduce the frequency of migraines and corresponding acute neurostimulation treatments. In particular, the patient may determine that the average dose per treatment of about 4.9 cC should be gradually increased by increasing the average treatment duration of 10 minutes and 54 seconds. Before or during a preventative treatment session, the patient may modify the treatment duration to a predetermined amount (e.g., 15 minutes) to generate a third (e.g., customized, user-defined) treatment program. The third treatment program may enable a user to set their own intensity pathway and function as a customized preventative treatment program or a customized acute treatment program.

In particular, referring to the GUIs 1803, 1806, 1807, 1808, 1810, 1811, a maintain intensity icon 1848 may be selected to increase a stimulation parameter (e.g., treatment duration) of a predetermined treatment program to generate a new (e.g., patient defined) treatment program that may be subsequently re-applied for future treatments, and which may be further modified as desired. A modifiable treatment program may encourage patient compliance with neurostimulation treatment and may improve patient outcomes. It should be appreciated that the user may generate any number of modified treatment programs (e.g., third treatment program, fourth treatment program, fifth treatment program, sixth treatment program, etc.).

In some variations, the customized (e.g., third) treatment program may be selectable from a GUI similar to the GUI 1802, but having a third option for the third treatment program. For example, a patient may subsequently select the third treatment program having a first predetermined treatment duration (e.g., 15 minutes) until the patient further modifies the treatment program (e.g., increases the treatment duration to 20 minutes). For example, the patient may generate a fourth treatment program having a second predetermined treatment duration (e.g., 20 minutes) when the patient becomes comfortable with (e.g., accustomed to) the first predetermined treatment duration (e.g., 15 minute) preventative treatment session duration. Similarly, the patient may determine that an acute treatment program session having a higher dose and lower duration may be more effective in providing acute migraine relief and may generate a fifth treatment program reflecting such modifications.

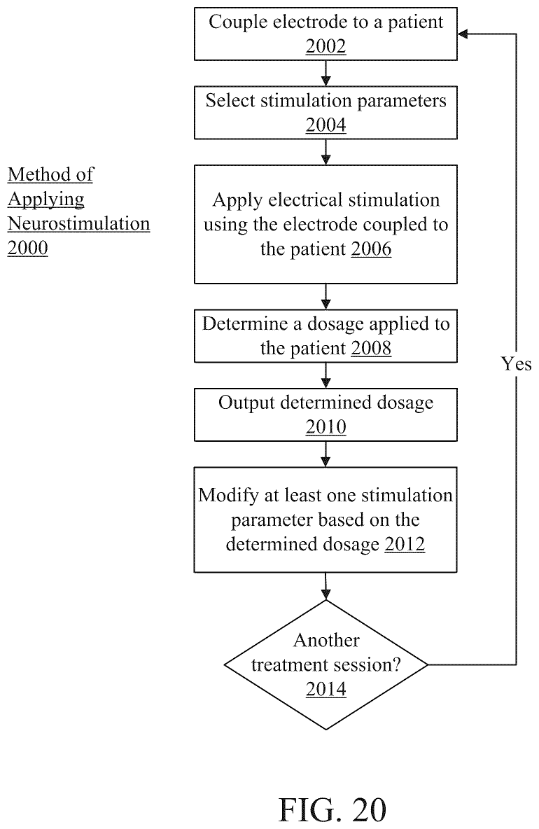

A method of applying neurostimulation treatment may include selecting a neurostimulation treatment program having a set of stimulation parameters, applying electrical stimulation using the electrode coupled to the patient, determining a neurostimulation dosage applied to the patient, and modifying at least one stimulation parameter based on the determined dosage. For example, FIG. 20 depicts a flowchart representation that generally describes a method of applying transcutaneous electrical stimulation to a patient 2000 using any of the systems and devices described herein. The method 2000 may include coupling an electrode to the forehead of a patient 2002. For example, an electrode 204 may be coupled to a patient 250 (e.g., forehead of the patient).

In some variations, a set of stimulation parameters may be selected for stimulating an anatomical target (e.g., a nerve such as a trigeminal nerve) of the patient 2004. The set of stimulation parameters may correspond to a treatment program such as a preventative treatment program having a first set of stimulation parameters configured to preemptively treat a disorder, an acute treatment program having a second set of stimulation parameters configured to acutely treat the disorder, or another (e.g., customized, modified) treatment program. For example, a first treatment program (e.g., preventative treatment program) may be configured to preemptively treat a disorder (e.g., migraine), a second treatment program (e.g., acute treatment program) may be configured to acutely treat the disorder, and a third treatment program (e.g., user-defined intensity pathway) may be customized by a user. As described in more detail herein, the third treatment program may correspond to a user-selected set of stimulation parameters (e.g., customized preventative treatment program, customized acute treatment program). In some variations, the set of stimulation parameters may be selected using a GUI such as shown in GUIs 1700, 1710, 1800, 1801, 1802, 1803, 1805, 1806, 1807, 1808, 1809, 1810, 1811.

In some variations, a stimulation parameter may be one or more of a frequency, a current, a pulse width, a pulse amplitude, a dead time, a pulse duration, a session time, a session duration, a maximum current amplitude in a session, and a session frequency. For example, in some variations, the frequency may be between about 10 Hz and about 300 Hz, the current may be between about 1 mA and about 35 mA, the pulse width may be between about 240 μs and about 260 μs, the pulse amplitude may be up to about 17 mA, the dead time may be between about 1 μs and about 10 μs, the duration may be between about 150 microseconds and about 450 microseconds, and a maximum increase in current of may be up to about 20 mA at a rate of less than or equal to about 40 microamperes per second and with a step up in current not exceeding about 50 microamperes.

In some variations, electrical stimulation having the selected stimulation parameters may be applied using an electrode coupled to the patient 2006. For example, in variations in which the electrode is coupled to a forehead of the patient, applying the electrical stimulation may include stimulating an afferent path of a supratrochlear nerve and an afferent path of a supraorbital nerve of an ophthalmic branch of the trigeminal nerve. A set of pulses for the electrode applying the electrical stimulation may be generated using a signal generator. The electrical stimulation may be configured to treat one or more of migraines, tension, headaches, cluster headaches, hemicrania continua, Semi Unilateral Neuralgaform Non Conjunctival Tearing (SUCNT), chronic paroxystic hemicranias, trigeminal neuralgia, facial nerve disturbances, autism, depression, cyclothymia, coma, anxiety, tremor, aphasia, insomnia, sleep disorders, hypersomnia, epilepsy, attention deficit hyperactivity disorder, Parkinson's disease, Alzheimer's disease, multiple sclerosis, stroke, and Cerebellar syndrome. As described herein, one or more stimulation parameters may be modified while applying the electrical stimulation (e.g., during a treatment session). For example, a patient may provide input to a GUI to increase a stimulation duration or maximum current amplitude.

In some variations, a dosage of the electrical stimulation applied to the trigeminal nerve may be determined 2008. For example, dosage determination may include calculating an electric charge delivered to the trigeminal nerve of the patient. For example, an electric charge delivered may be determined by calculating current (e.g., pulse amplitude) multiplied by duration (e.g., pulse width) over a session duration. In some variations, the determined dosage may be output 2010. For example, a graphical user interface comprising the determined dosage may be generated and displayed on a computing device.

In some variations, at least one stimulation parameter may be modified based on the determined dosage 2012. For example, a first treatment program session frequency and/or dosage may be increased based on the determined dosage and/or a second treatment program session frequency may be reduced. In some variations, applying the electrical stimulation may include modifying at least one stimulation parameter during one of the first treatment session and the second treatment session to generate a third treatment program having a third set of stimulation parameters. For example, the third treatment program may enable a user to set their own intensity pathway and function as a customized preventative treatment program or a customized acute treatment program.

Optionally, a determination may be performed of whether to initiate another treatment session 2014. If so, the process may return to step 2002 where selecting stimulation parameters 2004 may include selecting the third treatment program having the third set of stimulation parameters. As described herein, as a result of modifying at least one stimulation parameter, the dosage may be reduced over subsequent time periods.

### Exemplary Embodiments

Embodiment A1. A system for applying transcutaneous electrical stimulation to a patient, comprising:
an electrode configured to be coupled to a forehead of the patient, the electrode comprising:
   a substrate;
   a first conductor, a second conductor, and an insulator each disposed on the substrate, the insulator positioned laterally between the first and second conductor, the first and second conductors configured to stimulate a trigeminal nerve of the patient; and
   an electrode identifier disposed on the substrate and across the first and second conductors; and
   a housing configured to releasably couple to the electrode, the housing comprising a signal generator configured to generate a set of pulses for the electrode.

Embodiment A2. The system of Embodiment A1, wherein the electrode identifier comprises at least two apertures, and a first magnet coupled to a first adhesive conductor, and a second magnet coupled to a second adhesive conductor, wherein the first and second magnets project through a respective aperture of the electrode identifier.

Embodiment A3. The system of Embodiment A2, wherein the housing is configured to be coupled to the forehead of the patient using the electrode.

Embodiment A4. The system of Embodiment A2, wherein the housing is configured to magnetically couple to the first and second magnets of the electrode.

Embodiment A5. The system of Embodiment A2, wherein the first and second magnets are configured to receive the set of pulses generated by the signal generator.

Embodiment A6. The system of Embodiment A2, wherein the electrode identifier defines a third aperture and the insulator defines a fourth aperture corresponding to the third aperture of the insulator.

Embodiment A7. The system of Embodiment A1, wherein the electrode identifier overlaps the first and second conductors.

Embodiment A8. The system of Embodiment A1, wherein the electrode identifier is disposed on the insulator.

Embodiment A9. The system of Embodiment A1, wherein the electrode identifier overlaps the insulator.

Embodiment A10. The system of Embodiment A1, wherein the electrode identifier comprises a Radio Frequency Identification (RFID) tag.

Embodiment A11. The system of Embodiment A1, wherein the insulator separates the first conductor from the second conductor.

Embodiment A12. The system of Embodiment A1, wherein the electrode comprises an adhesive conductor area of the first and second adhesive conductor between about 50% and about 80% of a substrate area of the substrate.

Embodiment A13. The system of Embodiment A1, wherein the first conductor comprises a first lateral end opposite the insulator, and the second conductor comprises a second lateral end opposite the insulator.

Embodiment A14. The system of Embodiment A13, wherein the first lateral end, the second lateral end, and the insulator are non-overlapping with the first and second adhesive conductors.

Embodiment A15. The system of Embodiment A13, wherein the first lateral end and the second lateral end comprise a lateral end area of up to about 20% of a substrate area of the substrate.

Embodiment A16. The system of Embodiment A13, wherein each of the first conductor and the second conductor tapers from the insulator to the respective lateral end.

Embodiment A17. The system of Embodiment A1, wherein the signal generator is configured to generate the set of pulses comprising a current of between about 1 mA and about 35 mA.

Embodiment A18. The system of Embodiment A1, wherein the signal generator is configured to generate the set of pulses comprising a pulse width between about 240 μs and about 260 μs, a pulse amplitude of up to about 17 mA, and a dead time of between about 1 μs and about μs.

Embodiment A19. The system of Embodiment A1, wherein the signal generator is configured to generate the set of pulses comprising a duration of between about 150 microseconds and about 450 microseconds with a maximum increase in current of up to about 20 mA at a rate of less than or equal to about 40 microamperes per second and with a step up in current not exceeding about 50 microamperes.

Embodiment A20. The system of Embodiment A1, wherein the electrode is configured to stimulate an afferent path of a supratrochlear nerve and an afferent path of a supraorbital nerve of an ophthalmic branch of the trigeminal nerve.

Embodiment A21. The system of Embodiment A1, wherein the substrate comprises a length of between about 70 mm and about 120 mm.

Embodiment A22. The system of Embodiment A1, wherein the insulator comprises a length of between about 15 mm and about 50 mm, and a width of between about 5 mm and about 15 mm.

Embodiment A23. The system of Embodiment A13, wherein the first lateral end and the second lateral end each comprise a height of between about 5 mm and about 20 mm, and a width of between about 5 mm and about 10 mm.

Embodiment A24. The system of Embodiment A1, further comprising a power source, wherein the housing is configured to separate the power source from the signal generator.

Embodiment A25. The system of Embodiment A24, wherein the signal generator is separated from the power source by a predetermined distance.

Embodiment A26. The system of Embodiment A24, wherein the housing comprises a set of protrusions configured to separate the power source from the signal generator.

Embodiment A27. The system of Embodiment A24, wherein the power source comprises a battery.

Embodiment A28. The system of Embodiment A1, wherein the housing comprises a power source coupled to the signal generator, and further comprising a charger configured to wirelessly charge the power source.

Embodiment B1. An electrode configured to be coupled to a forehead of the patient, the electrode comprising:
a substrate;
a first conductor, a second conductor, and an insulator each disposed on the substrate, the insulator positioned laterally between the first and second conductor, the first and second conductors configured to stimulate a trigeminal nerve of the patient; and
an electrode identifier disposed on the substrate and across the first and second conductors.

Embodiment C1. A system, comprising:
a signal generator configured to generate a set of pulses for transcutaneous stimulation of a trigeminal nerve of a patient;
an identifier reader configured to detect an electrode identifier of an electrode releasably coupled to the system; and
a processor and a memory coupled to the identifier reader, the processor configured to:
   detect the electrode identifier using the identifier reader;
   generate an authentication signal based on the detected identifier; and
   stimulate the trigeminal nerve of the patient using the set of pulses based on the authentication signal.

Embodiment C2. The system of Embodiment C1, wherein the processor is configured to:
inhibit generation of the set of pulses when the electrode identifier is not detected.

Embodiment C3. The system of Embodiment C1, wherein the processor is configured to:
inhibit generation of the set of pulses when the authentication signal is one or more of unauthorized, expired, and overused.

Embodiment D1. A method of treating a patient, comprising:
coupling an electrode to a forehead of the patient, the electrode comprising an electrode identifier for the electrode;
coupling a housing of an electrical stimulation system to the electrode, the electrical stimulation system comprising a signal generator and an identifier reader;
detecting the electrode identifier using the identifier reader of the electrical stimulation system;
generating an authentication signal based on the detected identifier; and
stimulating a trigeminal nerve of the patient using a set of pulses generated by the signal generator based on the authentication signal.

Embodiment D2. The method of Embodiment D1, wherein the stimulating is configured to treat one or more of migraine, tension, headaches, cluster headaches, hemicrania continua, Semi Unilateral Neuralgaform Non Conjunctival Tearing (SUCNT), chronic paroxystic hemicranias, trigeminal neuralgia, facial nerve disturbances, autism, depression, cyclothymia, coma, anxiety, tremor, aphasia, insomnia, sleep disorders, hypersomnia, epilepsy, attention deficit hyperactivity disorder, Parkinson's disease, Alzheimer's disease, multiple sclerosis, stroke, and Cerebellar syndrome.

Embodiment D3. The method of Embodiment D1, further comprising releasing the system from the electrode.

Embodiment D4. The method of Embodiment D1, further comprising storing one or more of a session time, a treatment stimulation program selected, a session duration, a maximum current amplitude in a session, a session error, a number of repetitions, a sum of current delivered, a sum of current delivered if maximum current amplitude was reached, a battery charge time, a battery charge duration, a duration to reach full charge, and a battery charge error.

Embodiment E1. A system for applying transcutaneous electrical stimulation to a patient, comprising:
an electrode configured to be coupled to a forehead of the patient, the electrode comprising:
   a substrate;
   a first conductor, a second conductor, and an insulator each disposed on the substrate, the insulator positioned laterally between the first and second conductor, the first and second conductors configured to stimulate a trigeminal nerve of the patient; and
   a housing configured to allow for dimensional changes in the power source by separating the power source from the signal generator.

Embodiment E2. The system of Embodiment E1, wherein the housing is configured to releasably couple to the electrode, the housing comprising:
a signal generator configured to generate a set of pulses for the electrode;
a power source coupled to the signal generator;
a set of protrusions configured to separate the power source from the signal generator by a first predetermined distance.

Embodiment E3. The system of Embodiment E1, wherein the set of protrusions comprises a set of fasteners.

Embodiment E4. The system of Embodiment E3, wherein the set of protrusions are configured to couple to a first side of the signal generator and the set of fasteners are configured to couple to a second side of the signal generator opposite the first side.

Embodiment E5. The system of Embodiment E3, wherein the set of fasteners comprises a first fastener and a second fastener separated by a distance between about 25 mm and about 35 mm.

Embodiment E6. The system of Embodiment E1, wherein the power source is configured to increase in dimension within the housing when the set of pulses are generated.

Embodiment E7. The system of Embodiment E1, wherein the power source comprises a battery.

Embodiment E8. The system of Embodiment E1, wherein the set of protrusions are configured to attach the signal generator to the housing.

Embodiment E9. The system of Embodiment E1, wherein the housing comprises a connector configured to releasably couple to the electrode, the set of protrusions configured to separate the signal generator from the connector by a second predetermined distance.

Embodiment E10. The system of Embodiment E1, further comprising a charger configured to wirelessly charge the power source.

Embodiment E11. The system of Embodiment E1, further comprising an electrode identifier disposed on the substrate.

Embodiment F1. A method for applying transcutaneous electrical stimulation to a patient, comprising:
selecting one or more stimulation parameters for the electrical stimulation;
applying the electrical stimulation having the selected one or more stimulation parameters using an electrical stimulation system coupled to the patient;
determining a dosage of the electrical stimulation applied to patient; and modifying at least one stimulation parameter based on the determined dosage.

Embodiment F2. The method of Embodiment F1, wherein determining the dosage comprises calculating an electric charge delivered to the patient by the electrical stimulation system.

Embodiment F3. The method of Embodiment F1, wherein selecting the one or more stimulation parameters comprises selecting one of a first treatment program having a first set of stimulation parameters and configured to preemptively treat a disorder and a second treatment program having a second set of stimulation parameters and configured to acutely treat the disorder.

Embodiment F4. The method of claim F3, wherein modifying the at least one stimulation parameter based on the determined dosage comprises increasing a first treatment program session frequency and reducing a second treatment program session frequency.

Embodiment F5. The method of claim F3, wherein modifying the at least one stimulation parameter based on the determined dosage results in increasing the dosage of the first treatment program.

Embodiment F6. The method of claim F4, further comprising reducing the dosage over a predetermined time period after modifying the at least one stimulation parameter.

Embodiment F7. The method of claim F3, wherein a stimulation parameter of the one or more stimulation parameters is adjusted while applying the electrical stimulation.

Embodiment F8. The method of claim F7, further comprising generating a third treatment program having a third set of stimulation parameters based on the adjusted stimulation parameter.

Embodiment F9. The method of claim F8, wherein selecting the one or more stimulation parameters comprises selecting the third treatment program.

Embodiment F10. The method of Embodiment F1, further comprising generating a graphical user interface comprising the determined dosage.

Embodiment F11. The method of Embodiment F1, wherein the one or more stimulation parameters comprise one or more of a frequency, a current, a pulse width, a pulse amplitude, a dead time, a pulse duration, a session time, a session duration, a maximum current amplitude in a session, and a session frequency.

Embodiment F12. The method of Embodiment F1, wherein the electrical stimulation comprises a frequency of the electrical stimulation, wherein the frequency is between about 10 Hz and about 300 Hz.

Embodiment F13. The method of Embodiment F1, wherein the electrical stimulation comprises a current of between about 1 mA and about 35 mA.

Embodiment F14. The method of Embodiment F1, wherein the electrical stimulation comprises a pulse width between about 240 μs and about 260 μs.

Embodiment F15. The method of Embodiment F1, wherein the electrical stimulation comprises a pulse amplitude of up to about 17 mA.

Embodiment F16. The method of Embodiment F1, wherein the electrical stimulation comprises a dead time of between about 1 μs and about 10 μs.

Embodiment F17. The method of Embodiment F1, wherein the electrical stimulation comprises a duration of between about 150 microseconds and about 450 microseconds with a maximum increase in current of up to about 20 mA at a rate of less than or equal to about 40 microamperes per second and with a step up in current not exceeding about 50 microamperes.

Embodiment F18. The method of Embodiment F1, wherein applying the electrical stimulation comprises stimulating an afferent path of a supratrochlear nerve and an afferent path of a supraorbital nerve of an ophthalmic branch of a trigeminal nerve.

Embodiment F19. The method of Embodiment F1, wherein the electrical stimulation system comprises a signal generator releasably coupled to an electrode, and wherein applying the electrical stimulation comprises generating a set of pulses for the electrode using the signal generator.

Embodiment F20. The method of Embodiment F1, wherein applying the electrical stimulation treats one or more of: a migraine, tension, headaches, cluster headaches, hemicrania continua, Semi unilateral neuralgaform non conjunctival tearing (SUCNT), chronic paroxystic hemicranias, trigeminal neuralgia, facial nerve disturbances, autism, depression, cyclothymia, coma, anxiety, tremor, aphasia, insomnia, sleep disorders, hypersomnia, epilepsy, attention deficit hyperactivity disorder, Parkinson's disease, Alzheimer's disease, multiple sclerosis, stroke, and Cerebellar syndrome.

Embodiment G1. An electrical stimulation system, comprising:
an electrode configured to be coupled to a patient;
a signal generator operably coupled to the electrode and configured to generate a set of pulses for transcutaneous electrical stimulation of the patient; and
a processor and a memory coupled to the signal generator, the processor configured to:
   receive one or more stimulation parameters;
   apply the electrical stimulation having the received one or more stimulation parameters to a nerve of a patient using the electrode;
   determine a dosage of the electrical stimulation applied to the nerve; and
   receive at least one modified stimulation parameter based on the determined dosage.

Embodiment G2. The system of Embodiment G1, wherein determining the dosage comprises calculating an electric charge delivered to the nerve of the patient.

Embodiment G3. The system of Embodiment G1, wherein receiving the one or more stimulation parameters comprises selecting one of a first treatment program having a first set of stimulation parameters and configured to preemptively treat a disorder and a second treatment program having a second set of stimulation parameters and configured to acutely treat the disorder.

Embodiment G4. The system of Embodiment G3, wherein the processor is configured to receive an increase in a first treatment program session frequency based on the determined dosage and a reduction in a second treatment program session frequency.

Embodiment G5. The system of Embodiment G3, wherein the processor is configured to receive an increase in the dosage of the first treatment program.

Embodiment G6. The system of Embodiment G4, wherein the processor is configured to receive a reduction in the dosage of the first treatment program over a predetermined time period after modifying the at least one stimulation parameter.

Embodiment G7. The system of Embodiment G3, wherein the processor is configured to receive at least one modified stimulation parameter during one of the first treatment session and the second treatment session.

Embodiment G8. The system of Embodiment G7, wherein the processor is configured to generate a third treatment program having a third set of stimulation parameters based on the received at least one modified stimulation parameters during one of the first treatment session and the second treatment session.

Embodiment G9. The system of Embodiment G8, wherein the processor is configured to receive a selection of the third treatment program.

Embodiment G10. The system of Embodiment G1, wherein the processor is configured to generate a graphical user interface comprising the determined dosage.

Embodiment G11. The system of Embodiment G1, wherein the electrical stimulation comprise one or more of a frequency, a current, a pulse width, a pulse amplitude, a dead time, a pulse duration, a session time, a session duration, a maximum current amplitude in a session, and a session frequency.

Embodiment G12. The system of Embodiment G1, wherein the electrical stimulation comprises a frequency of between about 10 Hz and about 300 Hz.

Embodiment G13. The system of Embodiment G1, wherein the electrical stimulation comprises a current of between about 1 mA and about 35 mA.

Embodiment G14. The system of Embodiment G1, wherein the electrical stimulation comprises a pulse width between about 240 μs and about 260 μs.

Embodiment G15. The system of Embodiment G1, wherein the electrical stimulation comprises a pulse amplitude of up to about 17 mA.

Embodiment G16. The system of Embodiment G1, wherein the electrical stimulation comprises a dead time of between about 1 μs and about 10 μs.

Embodiment G17. The system of Embodiment G1, wherein the electrical stimulation comprises a duration of between about 150 microseconds and about 450 microseconds with a maximum increase in current of up to about 20 mA at a rate of less than or equal to about 40 microamperes per second and with a step up in current not exceeding about 50 microamperes.

Although the foregoing variations have, for the purposes of clarity and understanding, been described in some detail by illustration and example, it will be apparent that certain changes and modifications may be practiced, and are intended to fall within the scope of the appended claims. Additionally, it should be understood that the components and characteristics of the systems and devices described herein may be used in any combination. The description of certain elements or characteristics with respect to a specific figure are not intended to be limiting or nor should they be interpreted to suggest that the element cannot be used in combination with any of the other described elements. For all of the variations described herein, the steps of the methods may not be performed sequentially. Some steps are optional such that every step of the methods may not be performed.

1.-66. (canceled)

67. An electrical stimulation system, comprising:
an electrode configured to be coupled to a patient;
a signal generator operably coupled to the electrode and configured to generate a set of pulses for transcutaneous electrical stimulation of the patient; and
a processor and a memory coupled to the signal generator, the processor configured to:
receive one or more stimulation parameters;
apply the electrical stimulation having the received one or more stimulation parameters to a nerve of a patient using the electrode;
determine a dosage of the electrical stimulation applied to the nerve; and
receive at least one modified stimulation parameter based on the determined dosage.

68. The system of claim 67, wherein determining the dosage comprises calculating an electric charge delivered to the nerve of the patient.

69. The system of claim 67, wherein receiving the one or more stimulation parameters comprises selecting one of a first treatment program having a first set of stimulation parameters and configured to preemptively treat a disorder and a second treatment program having a second set of stimulation parameters and configured to acutely treat the disorder.

70. The system of claim 69, wherein the processor is configured to receive an increase in a first treatment program session frequency based on the determined dosage and a reduction in a second treatment program session frequency.

71. The system of claim 69, wherein the processor is configured to receive an increase in the dosage of the first treatment program.

72. The system of claim 70, wherein the processor is configured to receive a reduction in the dosage of the first treatment program over a predetermined time period after modifying the at least one stimulation parameter.

73. The system of claim 69, wherein the processor is configured to receive at least one modified stimulation parameter during one of the first treatment session and the second treatment session.

74. The system of claim 73, wherein the processor is configured to generate a third treatment program having a third set of stimulation parameters based on the received at least one modified stimulation parameters during one of the first treatment session and the second treatment session.

75. The system of claim 74, wherein the processor is configured to receive a selection of the third treatment program.

76. The system of claim 67, wherein the processor is configured to generate a graphical user interface comprising the determined dosage.

77. The system of claim 67, wherein the electrical stimulation comprise one or more of a frequency, a current, a pulse width, a pulse amplitude, a dead time, a pulse duration, a session time, a session duration, a maximum current amplitude in a session, and a session frequency.

78. The system of claim 67, wherein the electrical stimulation comprises a frequency of between about 10 Hz and about 300 Hz.

79. The system of claim 67, wherein the electrical stimulation comprises a current of between about 1 mA and about 35 mA.

80. The system of claim 67, wherein the electrical stimulation comprises a pulse width between about 240 μs and about 260 μs.

81. The system of claim 67, wherein the electrical stimulation comprises a pulse amplitude of up to about 17 mA.

82. The system of claim 67, wherein the electrical stimulation comprises a dead time of between about 1 μs and about 10 μs.

83. The system of claim 67, wherein the electrical stimulation comprises a duration of between about 150 microseconds and about 450 microseconds with a maximum increase in current of up to about 20 mA at a rate of less than or equal to about 40 microamperes per second and with a step up in current not exceeding about 50 microamperes.
